(19) United States
(12) Patent Application Publication
(10) Pub. No.: US 2009/0011400 A1
(43) Pub. Date: Jan. 8, 2009

(54) Substrate switched ammonia lyases and mutases

(75) Inventors: Joseph P. Noel, San Diego, CA (US); Gordon V. Louie, San Diego, CA (US); Marianne E. Bowman, San Diego, CA (US); Bradley S. Moore, La Jolla, CA (US); Michelle C. Moffitt, Lilli Pilli (AU)

Correspondence Address: QUINE INTELLECTUAL PROPERTY LAW GROUP, P.C., P O BOX 458, ALAMEDA, CA, 94501, US

(73) Assignee: The Salk Institute for Biological Studies and The Regents of the University of California

(21) Appl. No.: 11/998,828

(22) Filed: Nov. 30, 2007

Related U.S. Application Data
(60) Provisional application No. 60/872,162, filed on Dec. 1, 2006.
(60) Provisional application No. 60/873,668, filed on Dec. 6, 2006.
(60) Provisional application No. 60/874,709, filed on Dec. 12, 2006.

(51) Int. Cl. C12Q 1/00; C12N 9/00; C12N 15/11; C12N 1/20; C12N 1/19; C12N 5/04; C12N 5/06; C40B 40/10; G06G 7/50

(52) U.S. Cl. 435/4; 435/183; 536/23.2; 435/252.3; 435/254.11; 435/419; 435/325; 506/18; 703/11

(57) ABSTRACT

Crystal structure information is used to make substrate-switched amino acid ammonia lyase enzymes, including TALs, PALs and HALs. Related methods, systems, compositions, cells and transgenic organisms are provided.

### CROSS-REFERENCE TO RELATED APPLICATIONS

This application is related to and claims priority from the following applications: U.S. Ser. No. 60/872,162 SUBSTRATE SWITCHED AMMONIA LYSASES AND MUTASES by Noel et al., filed Dec. 1, 2006; U.S. Ser. No. 60/873,668 SUBSTRATE SWITCHED AMMONIA LYSASES AND MUTASES by Noel et al., filed Dec. 6, 2006; and U.S. Ser. No. 60/874,709 SUBSTRATE SWITCHED AMMONIA LYSASES AND MUTASES by Noel et al., filed Dec. 12, 2006. Each of these applications is incorporated herein by reference in their entirety.

### STATEMENT AS TO RIGHTS TO INVENTIONS MADE UNDER FEDERALLY SPONSORED RESEARCH AND DEVELOPMENT

This invention was made with government support under Grant No. MCB-0236027 from the National Science Foundation and support under Grant No. AI47818 from the National Institutes of Health. The government may have certain rights to this invention.

### FIELD OF THE INVENTION

The invention is in the field of protein engineering for production of phenylpropanoids and other compounds. Aromatic amino acid ammonia lyases such as phenylalanine ammonia lyase (PAL), tyrosine ammonia lyase (TAL) and histidine ammonia lyase (HAL) are engineered to switch substrates, permitting the rapid and efficient engineering of these lyases.

### BACKGROUND OF THE INVENTION

Phenylpropanoids constitute a large class of organic compounds that include lignins, stilbenes, and flavonoids, as just a few examples. Phenylpropanoids are synthesized by a broad range of naturally occurring organisms, including, for example, plants, fungi, and some bacteria, and demonstrate a variety of activities. For example, various phenylpropanoids play roles as antimicrobial agents, as feeding deterrents in defense against herbivores, and in UV protection. Phenylpropanoids are key constituents of various essential oils and are thus also of considerable commercial interest as fragrances and flavors. Phenylpropanoids such as isoflavonoids and stilbenes, which have been implicated as anticancer agents and in reduction of heart disease, respectively, are also of interest for their potential health benefits. Accordingly, there is considerable interest in metabolic engineering of phenylpropanoid synthetic pathways, e.g., for agricultural, nutritional, and medical purposes.

A number of enzymes in various phenylpropanoid biosynthetic pathways have been identified (see, e.g., Winkel-Shirley (2001) “Flavonoid biosynthesis: A colorful model for genetics, biochemistry, cell biology, and biotechnology” Plant Physiology 126:485-493). For example, the aromatic amino acid ammonia lyases phenylalanine ammonia lyase (PAL) and tyrosine ammonia lyase (TAL) catalyze the deamination of L-Phe and L-Tyr to produce the phenylpropanoid precursors cinnamic acid and coumaric acid, respectively. The ability to alter substrate specificity of these lyases would be desirable for phenylpropanoid pathway engineering. However, the determinants of substrate specificity of these amino acid lyases have not previously been fully defined.

The present invention overcomes these previous difficulties by providing structure-based methods of and models for modifying amino acid ammonia lyases to alter their substrate specificities, for example, for phenylpropanoid pathway engineering. These and other features of the invention will be apparent upon review of the following.

### SUMMARY OF THE INVENTION

The present invention includes the structural elucidation by crystallography of amino acid ammonia lyase enzymes, and the identification of those residues that are relevant for substrate specificity. Examples of mutations that switch substrate specificity are provided.

Thus, in a first aspect, the invention provides recombinant amino acid ammonia lyase enzymes, e.g., that include at least one mutation in an active site of the enzyme. The mutation switches substrate preference of the lyase enzyme from a first substrate to a second substrate. Most typically, the first substrate is an amino acid, and the second substrate is an amino acid; for example, the first and second amino acids are often aromatic amino acids. These can be naturally occurring common aromatic amino acids such as tyrosine, histidine or phenylalanine, or can be rare amino acids such as L-Dopa, or can be unnatural (e.g., synthetic) amino acids. In one example, the first amino acid is tyrosine or histidine and the second amino acid is phenylalanine. Similarly, the first amino acid can be phenylalanine and the second can be tyrosine or histidine. Type switching between tyrosine and histidine can also be performed.

In one example, the recombinant enzyme is derived from a tyrosine or histidine ammonia lyase, and preferentially deaminates L-Phe. For example, the mutation can be in a residue corresponding to His 89 of Rhodobacter sphaeroides Tyrosine Ammonia Lyase. This mutation switches the activity of the recombinant enzyme, as compared to the Rhodobacter sphaeroides Tyrosine Ammonia Lyase, from Tyrosine to phenylalanine. The recombinant amino acid ammonia lyase enzyme optionally comprises appropriate cofactors, such as a 4-methylidene-imidazole-5-one (MIO) cofactor prosthetic group.

In one desirable aspect, the recombinant enzyme produces trans-cinnamic acid. This is a useful intermediate in the synthesis of a variety of phenylpropanoids, e.g., lignins, flavonoids, stilbenes, coumarins, etc. The ability to easily engineer organisms (e.g., plants and microorganisms) for the production (or improved production) of phenylpropanoids is commercially valuable for the production of fragrances, flavorings, antibiotics, and many other valuable compounds.

Nucleic acids that encode recombinant amino acid ammonia lyase enzymes are an additional feature of the invention. These nucleic acids can be recombinant, synthetic, derived through mutation of natural nucleic acids, or the like. Recombinant cells that comprises the recombinant amino acid ammonia lyase enzyme or nucleic acid are also a feature of the invention. For example, the cell optionally encodes a recombinant tyrosine amino acid-type ammonia lyase enzyme that includes a mutation converting a kinetic preference of the enzyme for tyrosine into a preference for phenylalanine (or vice versa). The cell can be, e.g., a bacterial cell, a fungal cell, a plant cell or an animal cell. Desirably, the cell displays increased production of trans-cinnamic acid, or of a phenylpropanoid (e.g., lignins, flavonoids, stilbenes, coumarins, etc.), or both.

Additionally, knock-out and transgenic non-human animals comprising natural or recombinant ammonia lyase enzymes are a feature of the invention, e.g., to identify in vivo modulators of lyase activity and to analyze in vivo activity of the enzymes.

In a related aspect, the invention provides a library of amino acid ammonia lyase polypeptides. The library includes a plurality of polypeptides comprising or derived from amino acid ammonia lyase enzyme polypeptides. The plurality of polypeptides collectively comprise a plurality of mutations of at least one amino acid in at least one region of the polypeptides, corresponding to an active site of an amino acid ammonia lyase enzyme. All of the features described above with respect to the polypeptides, nucleic acids and cells are applicable to the libraries as well.

For example, the plurality of polypeptides are optionally derived from at least one tyrosine, phenylalanine, or histidine ammonia lyase enzyme. The plurality of mutations optionally include at least one mutation that switches a kinetic substrate preference of one or more of the polypeptides. The kinetic substrate preference is optionally switched from tyrosine or histidine to phenylalanine, or vice versa (or between tyrosine and histidine). The mutations optionally provide at least one residue that interacts with an aromatic ring of a substrate of the enzyme. The residue optionally corresponds to His 89 of RsTAL (e.g., a residue having the same structural relationship to the enzyme as His 89 does within RsTAL). Libraries of nucleic acids encoding the library of polypeptides, and libraries of cells that include the libraries of polypeptides are also a feature of the invention.

Methods of modifying an enzyme (e.g., an amino acid lyase or mutase enzyme) are also provided. The methods include accessing an information set derived from a crystal structure of an amino acid lyase enzyme, or of a homologue thereof, optionally complexed with a product. Based on information in the information set, the method includes predicting whether making a change to the structure of the enzyme will alter an interaction between a substrate, intermediate or product and the enzyme. The enzyme is modified based upon on the predictions made from the crystal structure information. Example crystal structure information, provided herein, includes the crystal structure of a tyrosine ammonia lyase enzyme, or a mutant thereof. For example, the information set can correspond to a crystal structure of a Rhodobacter sphaeroides tyrosine ammonia lyase enzyme, or a homologous variant thereof, complexed with cinnamate, caffeate, or coumarate. The tyrosine ammonia lyase enzyme can include a double homotetramer and optionally includes an MIO co-factor prosthetic group. The features described above for the compositions are applicable here as well.

Corresponding systems are also a feature of the invention. For example, an information storage module comprising an information set derived from a crystal structure of an amino acid ammonia lyase enzyme bound to a product is a feature of the invention.

In an additional aspect, the invention provides a method of deaminating L-DOPA. This includes contacting L-DOPA with a purified or recombinant tyrosine ammonia lyase enzyme. This invention provides the first description of L-DOPA deamination activity. The ability to deaminate L-DOPA has clinical relevance, e.g., in the treatment of Schizophrenia and Tourette's syndrome. Similarly, lowering peripheral L-DOPA levels is useful in L-DOPA mediated treatment of Parkinson's disease. Further, the product of the deamination of L-DOPA, caffeic acid, has been shown to have beneficial effects, including anti-tumor activity.

### BRIEF DESCRIPTION OF THE DRAWINGS

FIG. 1 schematically illustrates reactions catalyzed by the aromatic amino acid ammonia lyases and the related aminomutases. Tyrosine ammonia lyase (TAL), phenylalanine ammonia lyase (PAL), and histidine ammonia lyase (HAL) catalyze the non-oxidative deamination of their respective amino acid substrates, yielding the corresponding α-β unsaturated aryl-acid product plus ammonia. The aminomutases catalyze the α-β migration of the amino group of the α-amino acid substrate. Labeling experiments [1, 15] have shown that the brown β-proton (pro-S in L-Phe and L-Tyr, pro-R in L-His) of the substrate is stereospecifically abstracted. The dashed arrows emphasize the intermediacy of the lyase reaction in the overall reaction catalyzed by the aminomutases, which invoke Michael addition of ammonia to Cβ of the aryl-acid intermediate [1, 14-16].

FIG. 2 depicts the three-dimensional structure of RsTAL. FIG. 2A depicts ribbon representations of the RsTAL homotetramer, with the polypeptide chains of the individual monomers colored green (a), cyan (b), magenta (c), and yellow (d). The atoms of the four MIO co-factors are drawn as color-coded van der Waals spheres with red for oxygen, light gray for carbon and blue for nitrogen. Orthogonal views from the top (left) and front (right) of the homotetramer are shown. The 222 point-symmetry of the homotetramer is generated by three mutually orthogonal and intersecting two-fold rotational axes, shown as gray lines. In each orientation, two of the axes are visible with the third axis perpendicular to the page. FIG. 2B depicts a ribbon representation of the RsTAL monomer. The polypeptide chain is colored according to a gradient with blue and red serving as extremes for the N- and C-termini, respectively. The atoms of the MIO co-factor formed by the tripeptide segment Ala 149-Ser 150-Gly 151 are drawn as balls and sticks color-coded by atom type. The two-fold axes that relate this monomer to the other monomers in the homotetramer are shown as gray lines. FIG. 2C depicts electron density and interactions of the active-site lid loops of RsTAL complexed with coumarate, shown as a stereo pair. The three-stranded β-sheet is shown at the upper left. Three residues of the inner loop, Tyr 60, Phe 66, and Gly 67, encompass the bound coumarate product. Backbone hydrogen-bonding interactions of the lid loop are shown as magenta dashed lines; hydrogen-bonding interactions involving coumarate are represented as green dashed lines. The blue-colored contours envelope regions greater than 1.0σ in the final 2F_{obs}-F_{calc} electron-density map calculated at 1.58-Å resolution. FIG. 2D depicts the methylidene-imidazolone (MIO) co-factor. MIO and protein residues are shown as balls and sticks colored by atom type. Hydrogen-bonding interactions are represented as green dashed lines. An oxyanion hole is formed by the backbone amides of Leu 153 and Gly 204. The 149-150-151 numbering indicates the amino-acid origin of the MIO co-factor. The blue-colored contours envelope regions greater than 3σ in the MIO-omit F_{obs}-F_{calc} electron-density map. The inset shows the atom nomenclature of the native MIO cofactor, with the atom names colored according to atom type and numbered according to the originating residue within the 149-151 tripeptide (Ala 149 is 1, Ser 150 is 2 and Gly 151 is 3).

FIG. 3 depicts the active site of RsTAL. FIG. 3A shows a partial amino acid (single letter codes) sequence alignment of RsTAL with representative members of the aromatic amino acid ammonia lyase family discussed in the text. Only regions that form the active site of the enzymes are shown. Numbering is according to RsTAL. Yellow boxes highlight conserved catalytic and binding residues while the green box highlights the specificity determining residues. FIG. 3B depicts electron density and interactions of the coumarate product bound in the active site of wild-type RsTAL. The coumarate, MIO cofactor, and protein side-chains that line the active-site pocket are rendered as balls and sticks and colored according to atom type. Hydrogen-bonding interactions are shown as green dashed lines. The blue-colored contours envelope regions greater than 2.5σ in the initial F_{obs}—F_{calc} electron-density map calculated at 1.58-Å resolution with phases derived from the unliganded model. The closest atom of coumarate to the MIO co-factor (labels colored green) is labeled Cβ and colored red. FIG. 3C depicts electron density and interactions of the caffeate product bound in the active site of wild-type RsTAL. The phenyl ring of caffeate adopts primarily the conformation shown; a second, lower-occupancy conformation that differs only in the (inward) position of the meta-hydroxyl group is also observed. The blue-colored contours envelope regions greater than 2.5% in the initial F_{obs}-F_{calc} electron-density map calculated at 1.90-Å resolution with phases derived from the unliganded model. FIG. 3D depicts the product binding pocket in RsTAL. The depicted surface represents the area accessible to a probe sphere 1.4 Å in radius, and is color-coded according to the identity of the underlying protein atom (carbon is gray; nitrogen is blue; oxygen is red). The front portion of the RsTAL tetramer has been cut-away to reveal the internal cavity in the vicinity of the MIO co-factor. The coumarate molecule (shown in cyan) was excluded in the calculation of the molecular surface. The position of a caffeate molecule bound to RsTAL is shown in yellow. MIO is labeled (green) as shown in the inset of FIG. 2D.

FIG. 4 depicts product complexes of H89F RsTAL. FIG. 4A depicts electron density and interactions of the cinnamate product bound in the active site of H89F RsTAL. For the F_{obs}—F_{calc} electron-density map (1.9-Å resolution and shown contoured at 2.5σ), cinnamate and the side chain of Phe 89 were excluded from all calculations. FIG. 4B depicts electron density and interactions of coumarate bound to the H89F RsTAL active site. For the F_{obs}—F_{calc} electron-density map (2.0-Å resolution and shown contoured at 2.5σ), coumarate and the side chain of Phe 89 were excluded from all calculations. FIG. 4C depicts the chemical structure of 2-aminoindan-2-phosphonate (AIP). FIG. 4D depicts electron density and interactions of the PAL inhibitor AIP bound covalently to the MIO co-factor of H89F RsTAL. For the F_{obs}—F_{calc} electron-density map (1.75-Å resolution and shown contoured at 2.5σ), AIP, MIO and the side chain of Phe 89 were excluded from all calculations. The residue label shown in red, Asn 203, is involved in a backbone conformational rearrangement that allows the Asn 203 side-chain to engage the MIO co-factor upon AIP binding. FIG. 4E depicts a comparison of the binding modes of cinnamate (yellow), coumarate (orange-tan), and AIP (cyan) with the H89F RsTAL active site. Also shown is coumarate (magenta) bound to wild type RsTAL, with the hydrogen-bonding interactions between the coumarate product and wild type RsTAL represented as green dashed lines.

FIG. 5 depicts the active-site lid loops and a model for L-Tyr binding to RsTAL. FIG. 5A depicts the RsTAL homotetramer in the vicinity of the active-site pocket of monomer a in ribbon representation. The polypeptide chains of the individual monomers are colored as in FIG. 1A with the active-site lid loops shaded darker (green: inner loop of monomer a; yellow: outer loop of monomer d). The MIO co-factor, bound coumarate and protein residues that interact with the coumarate are drawn as balls and sticks and colored by atom type. FIG. 5B depicts a model for L-Tyr binding to RsTAL. The L-Tyr substrate (magenta) was modeled with minimal modifications from the binding mode of the coumarate product shown in FIG. 3B. FIG. 5C depicts a model for L-Tyr binding to RsTAL. The L-Tyr substrate was modeled based upon the binding mode of the AIP inhibitor shown in FIG. 4D, which places the α-amino group within covalent bonding distance of the Cβ2 methylidene carbon of the MIO cofactor (yellow dashed line). Note that a hydrogen bond between the L-Tyr-OH and the His 89-NE2 is preserved despite the shifted position of the L-Tyr substrate.

FIG. 6 provides a nucleic acid and protein sequence for RsTAL.

FIG. 7 provides a nucleic acid and protein sequence for an H89F mutant.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

An “amino acid ammonia lyase enzyme” is an enzyme that catalyzes the non-oxidative deamination of an amino acid substrate, yielding, e.g., the corresponding α-βunsaturated aryl-acid product plus ammonia.

A mutation “switches substrate preference” from a first substrate to a second substrate when the enzyme switches from displaying a kinetic preference for the first substrate to displaying a kinetic preference for the second substrate. Thus, by typical kinetic measurements such as Km, kcat and kcat/Km, the catalytic activity of the enzyme switches from a preference for the first substrate to a preference for the second substrate. Thus, for example, when the first and second substrate are present at equal concentrations (e.g., non-rate limiting concentrations) the enzyme will, after substrate preference is switched, convert the second substrate to product more rapidly and/or readily than it will convert the first substrate to a product. Examples of this switch include switching enzyme preference from a first amino acid to a second amino acid, e.g., a switch from preference for tyrosine or histidine to phenylalanine, or vice versa.

An “aminomutase” catalyzes the α-β migration of an amino group of an α-amino acid substrate.

A “rare” amino acid is a naturally occurring amino acid other than the common 20 amino acids that are typically incorporated into proteins during mRNA translation in a cell (an example genetic code listing the common 20 amino acids and the triplet nucleic acid codons that encode them is found in Stryer (1981) Biochemistry Second Edition W. H. Freeman and Company (New York), e.g., at p. 629). Examples of rare amino acids include selenocysteine and pyrolysine (which are optionally naturally incorporated into proteins by reprogramming of stop codons in certain organisms, but which, in other applications, are not incorporated into proteins), as well as amino acids such as L-3,4-dihydroxyphenylalanine (L-dopa), which, optionally, are not incorporated into proteins by the translational machinery of a cell (but which, optionally, can be incorporated, e.g., using artificial orthogonal translation components).

An “unnatural” amino acid is an amino acid that is not naturally occurring, produced, e.g., by synthetic or recombinant methods. A variety of unnatural amino acids, as well as methods of genetically encoding them into proteins, in vivo, using orthogonal tRNA-orthogonal aminoacyl synthetases are described in the literature. See, e.g., Wang and Schultz, “Expanding the Genetic Code,” Chem. Commun. (Camb.) 1:1-11 (2002); Wang and Schultz “Expanding the Genetic Code,” Angewandte Chemie Int. Ed., 44(1):34-66 (2005); Xie and Schultz, “An Expanding Genetic Code,” Methods 36(3):227-238 (2005); Xie and Schultz, “Adding Amino Acids to the Genetic Repertoire,” Curr. Opinion in Chemical Biology 9(6):548-554 (2005); Wang et al., “Expanding the Genetic Code,” Annu. Rev. Biophys. Biomol. Struct., 35:225-249 (2006; epub Jan. 13, 2006); and Xie and Schultz, “A chemical toolkit for proteins—an expanded genetic code,” Nat. Rev. Mol. Cell. Biol., 7(10):775-782 (2006; epub Aug. 23, 2006).

A second enzyme is “derived from” a first enzyme when the second enzyme (or coding nucleic acid thereof) is produced using sequence information from the first enzyme, or a coding nucleic acid thereof, or when the second enzyme (or coding nucleic acid thereof) is produced from the first enzyme (or coding nucleic acid thereof) by artificial, e.g., recombinant methods. For example, when the second enzyme is made by mutating a nucleic acid encoding the first enzyme, and expressing the resulting mutated nucleic acid, the second enzyme is said to be “derived from” the first enzyme. Similarly, when the second enzyme is made using sequence information from the first enzyme, e.g., by mutating the sequence of the first enzyme in silico and then synthesizing, e.g., a corresponding nucleic acid that encodes the second enzyme and expressing it, the resulting second enzyme is derived from the first enzyme.

An amino acid residue in a protein “corresponds” to a given residue when it occupies the same essential structural position within the protein as the given residue. For example, a selected residue in a selected protein corresponds to His 89 of Rhodobacter sphaeroides Tyrosine Ammonia Lyase when the selected residue occupies the same essential spatial or other structural relationship to other amino acids in the selected protein as His 89 does with respect to the other residues in Rhodobacter sphaeroides Tyrosine Ammonia Lyase. Thus, if the selected protein is aligned for maximum homology with the Rhodobacter sphaeroides Tyrosine Ammonia Lyase protein, the position in the aligned selected protein that aligns with His 89 is said to correspond to it. Instead of a primary sequence alignment, a three dimensional structural alignment can also be used, e.g., where the structure of the selected protein is aligned for maximum correspondence with the Rhodobacter sphaeroides Tyrosine Ammonia Lyase and the overall structures compared. In this case, an amino acid that occupies the same essential position as His 89 in the structural model is said to correspond to the His 89 residue.

A “library” of molecules, or a “molecular library” is a set of molecules. The molecules of the library optionally can be arranged for ease of access cataloguing, e.g., in one or more gridded arrays (e.g., in microtiter trays, gridded substrate libraries, or the like). Alternatively, the library can be arranged using more complex spatial relationships, e.g., using a computer system to track the relationship of the library members. The library can also include uncharacterized molecules, random molecules, or the like, where the spatial relationship of the library members is partially or completely unknown. Many libraries, e.g., expression libraries, lack fixed spatial relationships between the library members; in these formats, the library members can be deconvoluted by subcloning and/or dilution, e.g., after screening the library for an activity of interest.

An “information set derived from a crystal structure” is a set of information that includes crystal structure data, or which is derived from such data. For example, the information can take the form of atomic coordinates, mathematical transformations of such data, structural models that take account of such atomic coordinate information, or the like.

A “recombinant cell” is a cell that is made by artificial recombinant methods. The cell comprises one or more transgenes, e.g., heterologous amino acid ammonia lyase enzyme genes, introduced into the cell by artificial recombinant methods.

A “transgenic animal or plant” refers to a plant or animal that comprises within its cells a heterologous polynucleotide. In many embodiments, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant or animal part or plant or animal, the genotype of which has been altered by the presence of heterologous nucleic acid, including those transgenic organisms or cells initially so altered, as well as those created by crosses or asexual propagation from the initial transgenic organism or cell.

A variety of additional terms are defined or otherwise characterized herein.

### DETAILED DESCRIPTION

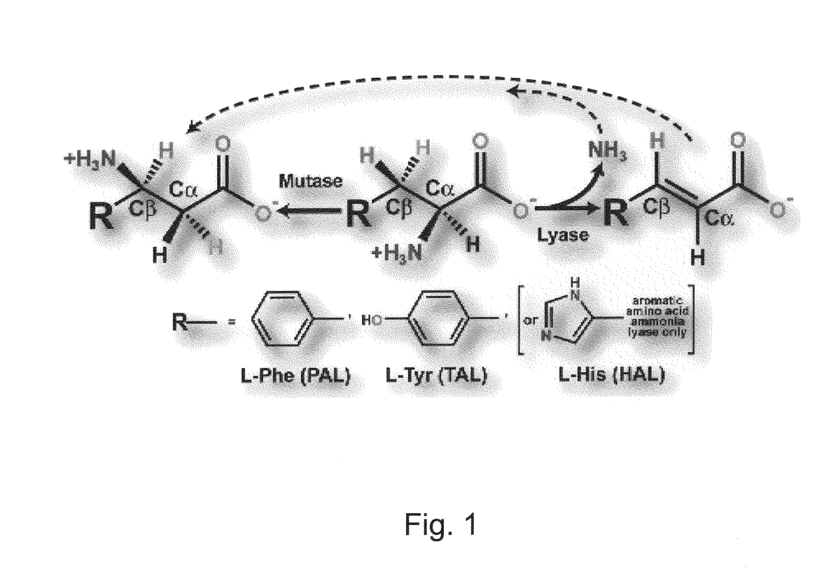

Tyrosine ammonia lyase (TAL) catalyzes the non-oxidative elimination of ammonia from L-Tyr, yielding trans-p-coumaric acid (trans-p-hydroxycinnamic acid). TAL is a member of a family of ammonia lyases that deaminate the aromatic amino acids, L-His, L-Phe, and L-Tyr (FIG. 1) [reviewed in 1; note: numbered references herein refer to the reference list at the end of the examples section below]. In plants and fungi, a dedicated TAL has not been identified, but instead phenylalanine ammonia lyase (PAL) occurs widely. PAL, which produces trans-cinnamic acid, catalyzes the committed step in a phenylpropanoid biosynthetic pathway leading to a variety of specialized phenolic plant and fungal metabolites. While PAL from dicotyledonous plants catalyzes the efficient deamination of L-Phe only, PAL from some monocots including maize efficiently deaminates both L-Phe and L-Tyr [2]. Similarly, PAL from the yeast Rhodosporidium toruloides turns over both L-Phe and L-Tyr [3]. Thus, PAL-derived TAL activity in monocots and fungi may provide an alternative route to the phenylpropanoid precursor p-coumaric acid, in lieu of hydroxylation of cinnamic acid by the membrane-bound cytochrome-P450 monooxygenase, cinnamate-4-hydroxylase.

In bacteria, phenylpropanoids are relatively rare, and, accordingly, PAL and TAL are poorly represented (at least based upon gene annotation). To date, PALs have been identified in Streptomyces maritimus [4], Photorhabdus luminescens [5], Sorangium cellulosum [6] and Streptomyces verticillatus [7]. In these bacteria, cinnamic acid serves as an intermediate in the biosynthesis of specific antibiotic or antifungal compounds (e.g., enterocin, 3,5-dihydroxy-4-isopropyl-stilbene, soraphen A and cinnamamide). PALs have also been recently identified in Anabaena variabilis and Nostoc punctiforme. The only confirmed sources of TAL are several species of purple phototropic bacteria (Rhodobacter capsulatus, Rhodobacter sphaeroides, and Halorhodospira halophila), in which p-coumarate is a precursor of the chromophore of photoactive yellow protein [8,9], and the actinomycete Saccharothrix espanaensis [10], in which coumarate is used for the biosynthesis of the saccharomicin antibiotics.

The aromatic amino-acid ammonia-lyases contain a 4-methylidene-imidazole-5-one (MIO) co-factor, formed by the spontaneous (autocatalytic) cyclization and dehydration of an internal Ala-Ser-Gly tripeptide segment [11]. Two alternative mechanisms have been suggested for the role of the electrophilic MIO co-factor in catalyzing the elimination of the α-amino group and the stereospecific abstraction of β-proton from the L-amino acid substrate [12, 13]. The earliest suggested mechanism invokes direct nucleophilic addition of the substrate's α-amino group to the exocyclic methylidene carbon of the MIO co-factor. A more recent proposal attempts to better rationalize the enhanced acidity of the substrate's β-proton, necessary for efficient deprotonation (normally the pKa of a benzyl proton is >40), and invokes attack by the electron-rich aromatic ring of the substrate through its δ-carbon (CD or C2 position relative to the 4-OH of L-Tyr) on the electron-deficient methylidene carbon in a Friedel-Crafts type mechanism [1]. While both mechanisms are intensely debated, both likely account for the activity of the recently characterized tyrosine- and phenylalanine aminomutases [14-16]. These MIO-dependent enzymes are closely related structurally and mechanistically to the aromatic amino acid ammonia-lyases forming intermediate aryl acids, but ultimately catalyze a second reactive step in which the α-amino group removed from the substrate is transferred to the β-carbon, yielding a β-amino acid product (FIG. 1).

Crystal structures are available for PALs from R. toruloides [13, 17] and Petroselinum crispum [18], as well as histidine ammonia lyase (HAL) from the bacterium Pseudomonas putida [19] (HAL deaminates histidine to urocanic acid, the first step in histidine catabolism). The structures demonstrate that the ammonia lyases share a common core three-dimensional structure. Nevertheless, despite the availability of these structures, the enzymatic mechanism and determinants of substrate specificity of the aromatic amino acid ammonia lyases have not previously been fully defined. Earlier studies have been hindered in part by a lack of accurate information pertaining to the mode of substrate/product binding.

We describe herein the crystal structure of TAL from the bacterium R. sphaeroides. The structure is the first for an aromatic amino acid ammonia lyase with a preference for L-Tyr as a substrate. We also describe the structures of TAL complexed with the products of the TAL-catalyzed reactions using L-Tyr and L-DOPA substrates, namely p-coumaric and caffeic acids, respectively. These structures provide the first definitive view of the binding of substrate or product to any aromatic amino acid ammonia lyase, thus identifying the substrate selectivity determinants of this family of enzymes. Based upon these high-resolution structures, RsTAL was successfully engineered into a kinetically authentic PAL, and additional structures of mutant RsTAL were obtained with cinnamic acid (the product of the PAL-catalyzed reaction using L-Phe as a substrate) and with the PAL-specific inhibitor 2-aminoindan-2-phosphonate (AIP).

Accordingly, the invention provides crystal structure information and mechanisms for using this information to engineer ammonia lyase enzymes to switch substrate specificity for these enzymes, including for TALs, PALs and HALs. Recombinant substrate-switched enzymes and coding nucleic acids, as well as cells that include the enzymes are features of the invention. Industrial and clinical aspects and applications for the invention include methods of phenylpropanoid synthesis, synthetic and clinical applications of this technology. Transgenic animals that comprise the relevant enzymes, nucleic acids and cells are also a feature of the invention. These and many other features are further described below and elsewhere herein. Details regarding aspects of the invention and disclosure herein can also be found in Louie et al. “Structural Determinants and Modulation of Substrate Specificity in Phenyalanine-Tyrosine Ammonia-Lyases” Chemistry and Biology 13, 1327-1338 (2006), incorporated herein by reference in its entirety.

The facility to modify substrate preference of a specific ammonia-lyase protein can also be useful for the exploitation of other useful properties of that specific protein. For instance, one may identify a particularly useful enzyme with good kinetic properties or in vivo stability properties, but lacking the substrate specificity desired. For instance, in treating PKU with PALs previous therapies have been constrained because many of the available PALs that are used are not stable in vivo, lack high activity and/or are problematic because they cause an immune response. In the present invention, because HALs are nearly ubiquitous in nature, one can select a HAL with desired properties (immunogenicity, in vivo stability, etc.) and convert it into a PAL using the methods herein.

### Generating and Using Crystal Structure Information for Modifying Enzymes

As is well-known in the art, the three-dimensional structures of proteins can be determined by x-ray crystallography. Typically, to determine the crystal structure of a protein, one or more crystals of the protein are obtained, diffraction data is collected from the crystals, and phases for the data are determined and used to calculate electron density maps in which a model of the protein is built. Additional rounds of model building and refinement can then be carried out to produce a reasonable model of the protein's structure. If desired, the structures of additional proteins can then be modeled based on homology with the protein whose structure has been determined.

Making Amino Acid Ammonia Lyase Crystals

Proteins are typically purified prior to crystallization, e.g., as described herein. Conditions for crystallizing proteins to obtain diffraction-quality crystals can be determined empirically using techniques known in the art. For example, crystallization conditions can be determined and optimized by screening a number of potential conditions, using vapor diffusion (e.g., hanging or sitting drop), microbatch, microdialysis, or similar techniques. Type and amount of precipitant (e.g., salt, polymer, and/or organic solvent), type and amount of additive, pH, temperature, etc. can be varied to identify conditions under which high quality crystals form. See, e.g., McPherson (1999) Crystallization of Biological Macromolecules Cold Spring Harbor Laboratory, Bergfors (1999) Protein Crystallization International University Line, Mullin (1993) Crystallization Butterwoth-Heinemann, Baldock et al. (1996) “A comparison of microbatch and vapor diffusion for initial screening of crystallization conditions” J. Crystal Growth 168:170-174, Chayen (1998) “Comparative studies of protein crystallization by vapor diffusion and microbatch” Acta Cryst. D54:8-15, Chayen (1999) “Crystallization with oils: a new dimension in macromolecular crystal growth” J. Crystal Growth 196:434-441, Page et al. (2003) “Shotgun crystallization strategy for structural genomics: an optimized two-tiered crystallization screen against the Thermotoga maritima proteome” Acta Crystallogr. D Biol. Crystallogr. 59:1028, Kimber et al. (2003) “Data mining crystallization databases: knowledge-based approaches to optimize protein crystal screens” Proteins 51:562, and Newman et al. (2005) “Towards rationalization of crystallization screening for small- to medium-sized academic laboratories: the PACT/JCG+strategy” Acta. Cryst. D61:1426.

Sparse matrix screening is described, e.g., in Jancarik and Kim (1991) “Sparse matrix sampling: a screening method for crystallization of proteins” J. Appl. Cryst. 24:409-411. Pre-formatted reagents for crystallization screening are commercially available, e.g., from Qiagen (www (dot) qiagen (dot) com) and Hampton Research (www (dot) hamptonresearch (dot) com). Screening is optionally automated, for example, using a robotic reagent dispensing platform.

Crystals of a complex, for example, an enzyme-product or enzyme-inhibitor complex, can be obtained by crystallizing the complex or by soaking crystals of the protein in a solution containing the product or inhibitor.

Specific examples of crystallization conditions for a tyrosine ammonia lyase and techniques for obtaining lyase-product and lyase-inhibitor complex crystals are described in the Examples sections below.

Crystal Structure Determination

Techniques for crystal structure determination are well known. See, for example, Stout and Jensen (1989) X-ray structure determination: a practical guide, 2nd Edition Wiley Publishers, New York; Ladd and Palmer (1993) Structure determination by X-ray crystallography, 3rd Edition Plenum Press, New York; Blundell and Johnson (1976) Protein Crystallography Academic Press, New York; Glusker and Trueblood (1985) Crystal structure analysis: A primer, 2nd Ed. Oxford University Press, New York; International Tables for Crystallography, Vol. F. Crystallography of Biological Macromolecules; McPherson (2002) Introduction to Macromolecular Crystallography Wiley-Liss; McRee and David (1999) Practical Protein Crystallography, Second Edition Academic Press; Drenth (1999) Principles of Protein X-Ray Crystallography (Springer Advanced Texts in Chemistry) Springer-Verlag; Fanchon and Hendrickson (1991) Crystallographic Computing, Volume 5 IUCr/Oxford University Press; and Murthy (1996) Crystallographic Methods and Protocols Humana Press.

In brief, once diffraction-quality crystals of the protein (e.g., unliganded or complexed with a substrate, intermediate or product) have been obtained, diffraction data is collected at one or more wavelengths. The wavelength at which the diffraction data is collected can be essentially any convenient wavelength. For example, data can be conveniently collected using an in-house generator with a copper anode at the CuKα wavelength of 1.5418 Å. Alternatively or in addition, data can be collected at any of a variety of wavelengths at a synchrotron or other tunable source. For example, data is optionally collected at a wavelength selected to maximize anomalous signal from the particular heavy atom incorporated in the protein, minimize radiation damage to the protein crystal, and/or the like.

The diffraction data is then processed and used to model the protein's structure. When the structure of a related protein is already known, the structure can be solved by molecular replacement. As another example, the protein can be derivatized with one or more heavy atoms to permit phase determination and structure solution, for example, by multiple isomorphous replacement (MIR), single isomorphous replacement (SIR), multiple isomorphous replacement with anomalous signal (MIRAS), single isomorphous replacement with anomalous signal (SIRAS), multiwavelength anomalous dispersion (MAD), or single wavelength anomalous dispersion (SAD) methods.

For example, in SAD phasing, the structure of the protein is determined by a process that comprises collecting diffraction data from the heavy atom-containing protein crystal at a single wavelength and measuring anomalous differences between Friedel mates, which result from the presence of the heavy atom in the crystal. In brief, collection of diffraction data involves measuring the intensities of a large number of reflections produced by exposure of one or more protein crystals to a beam of x-rays. Each reflection is identified by indices h, k, and 1. Typically, the intensities of Friedel mates (pairs of reflections with indices h,k,l and -h,-k,-l) are the same. However, when a heavy atom is present in the protein crystal and the wavelength of the x-rays used is near an absorption edge for that heavy atom, anomalous scattering by the heavy atom results in differences between the intensities of certain Friedel mates. These anomalous differences can be used to calculate phases that, in combination with the measured intensities, permit calculation of an electron density map into which a model of the protein structure can be built.

As another example, MAD phasing can be used. Here the structure of the protein is determined by a process that comprises collecting diffraction data from the heavy atom-containing protein crystal at two or more wavelengths and measuring dispersive differences between data collected at different wavelengths. For example, data is optionally collected at two wavelengths, e.g., at the point of inflection of the absorption curve of the heavy atom and at a remote wavelength away from the absorption edge, e.g., utilizing a synchrotron as the radiation source.

Suitable heavy atom derivatives for SIR, MIR, SAD, MAD, or similar techniques can be obtained when necessary by methods well known in the art. For example, crystals of the native protein can be soaked in solutions containing the desired heavy atom(s). As another example, heavy atom containing amino acids such as selenomethionine, selenocysteine, or telluromethionine can be incorporated into the protein before the protein is purified and crystallized. See, e.g., Dauter et al. (2000) “Novel approach to phasing proteins: derivatization by short cryo-soaking with halides” Acta Crystallogr D 56(Pt 2):232-237, Nagem et al. (2001) “Protein crystal structure solution by fast incorporation of negatively and positively charged anomalous scatterers” Acta Crystallogr D 57:996-1002), Boles et al. (1994) “Bio-incorporation of telluromethionine into buried residues of dihydrofolate reductase” Nat Struct Biol 1:283-284, Budisa et al. (1997) “Bioincorporation of telluromethionine into proteins: a promising new approach for X-ray structure analysis of proteins” J Mol Biol 270:616-623, and Strub et al. (2003) “Selenomethionine and selenocysteine double labeling strategy for crystallographic phasing” Structure 11: 1359-67.

A variety of programs to facilitate data collection, phase determination, model building and refinement, and the like are publicly available. Examples include, but are not limited to, the HKL2000 package (Otwinowski and Minor (1997) “Processing of X-ray Diffraction Data Collected in Oscillation Mode” Methods in Enzymology 276:307-326), the CCP4 package (Collaborative Computational Project (1994) “The CCP4 suite: programs for protein crystallography” Acta Crystallogr D 50:760-763), MOLREP (Vagin and Teplyakov (1997) “MOLREP: an automated program for molecular replacement” J. Appl. Crystallog. 30:1022-1025), SOLVE and RESOLVE (Terwilliger and Berendzen (1999) Acta Crystallogr D 55 (Pt 4):849-861), SHELXS and SHELXD (Schneider and Sheldrick (2002) “Substructure solution with SHELXD” Acta Crystallogr D Biol Crystallogr 58:1772-1779), Refmac5 (Murshudov et al. (1997) “Refinement of Macromolecular Structures by the Maximum-Likelihood Method” Acta Crystallogr D 53:240-255 and Vagin et al. (2004) Acta Crystallogr D Biol Crystallogr 60:2184-95), CNS (Brunger et al. (1998) Acta Crystallogr D Biol Crystallogr 54 (Pt 5):905-21), PRODRG (van Aalten et al. (1996) “PRODRG, a program for generating molecular topologies and unique molecular descriptors from coordinates of small molecules” J Comput Aided Mol Des 10:255-262), and O (Jones et al. (1991) “Improved methods for building protein models in electron density maps and the location of errors in these models” Acta Crystallogr A 47 (Pt 2): 110-119).

Specific examples of determination of the structures of wild-type and mutant amino acid ammonia lyases, amino acid ammonia lyase-product complexes, and an amino acid ammonia lyase-inhibitor complex are described in the Examples sections below.

Structure-Based Engineering of Amino Acid Ammonia Lyases and Other Enzymes

Structural data for an amino acid ammonia lyase or an amino acid ammonia lyase-product complex can be used to conveniently identify amino acid residues as candidates for mutagenesis to create variant enzymes having altered activities, for example, altered substrate preference or altered catalytic activity. For example, analysis of the three-dimensional structure of an amino acid ammonia lyase-product complex (e.g., a TAL-product complex) can identify residues that line the binding pocket of the active site, including residues that interact with the product and/or with a substrate; such residues can be mutated to modify substrate specificity of the enzyme (e.g., by adding or altering charge, hydrogen bonding potential, hydrophobicity, size, and/or the like). Similarly, residues can be identified that can be mutated to modify the catalytic activity of the enzyme.

The structure of a given amino acid ammonia lyase or amino acid ammonia lyase-product complex can be directly determined as described herein by x-ray crystallography or by NMR spectroscopy. Alternatively, the structure of an amino acid ammonia lyase or lyase-product complex can be modeled, for example, based on homology with an amino acid ammonia lyase or complex whose structure has already been determined (for example, any of the structures described herein in the Examples sections). A variety of programs to facilitate such homology modeling are publicly available, for example, MODELLER, which is commercially available from Accelrys (at www (dot) accelrys (dot) com) or on the internet at www (dot) salilab (dot) org/modeller; see Sali and Blundell (1993) “Comparative protein modelling by satisfaction of spatial restraints” J. Mol. Biol. 234:779-815 and Marti-Renom et al. (2000) “Comparative protein structure modeling of genes and genomes” Annu. Rev. Biophys. Biomol. Struct. 29:291-325.

The active site, including the binding pocket, of the amino acid ammonia lyase can be identified, for example, by examination of a lyase-product complex structure, homology with other amino acid ammonia lyases, biochemical analysis of mutant proteins, and/or the like. The position of a substrate or transition state intermediate (or a different product) in the binding pocket can be modeled, for example, by projecting the location of features of the substrate or intermediate (or other product) based on the previously determined location of a product in the binding pocket.

Such modeling of the substrate, intermediate, or product in the binding pocket of the amino acid ammonia lyase or a putative mutant amino acid ammonia lyase can involve simple visual inspection of a model of the amino acid ammonia lyase or lyase-product complex, for example, using molecular graphics software such as the PyMOL viewer (open source, freely available on the World Wide Web at www (dot) pymol (dot) org) or Insight II (commercially available from Accelrys at (www (dot) accelrys (dot) com/products/insight). Alternatively, modeling of the substrate, intermediate, or product in the binding pocket of the amino acid ammonia lyase or a putative mutant amino acid ammonia lyase, for example, can involve computer-assisted docking, molecular dynamics, free energy minimization, and/or like calculations. Such modeling techniques have been well described in the literature; see, e.g., Babine and Abdel-Meguid (eds.) (2004) Protein Crystallography in Drug Design, Wiley-VCH, Weinheim; Lyne (2002) “Structure-based virtual screening: An overview” Drug Discov. Today 7:1047-1055; Molecular Modeling for Beginners, at (www (dot) usm (dot) maine (dot) edu/˜rhodes/SPVTut/index (dot) html; and Methods for Protein Simulations and Drug Design at (www (dot) dddc dot) ac (dot) cn/embo04; and references therein. Software to facilitate such modeling is widely available, for example, the CHARMm simulation package, available academically from Harvard University or commercially from Accelrys (at www (dot) accelrys (dot) com), the Discover simulation package (included in Insight II, supra), and Dynama (available at (www (dot) cs (dot) gsu (dot) edu/˜cscrwh/progs/progs (dot) html). See also an extensive list of modeling software at (www (dot) netsci (dot) org/Resources/Software/Modeling/MMMD/top (dot) html.

Visual inspection and/or computational analysis of an amino acid ammonia lyase or an amino acid ammonia lyase-product complex model can identify relevant features of the enzyme that can be modified, including, for example, one or more residues that can be mutated to alter interaction between a substrate, intermediate, or product and the enzyme. For example, residues that form the active site binding pocket, including those that interact with the product in a lyase-product complex, are readily identified and can be mutated to alter product and/or substrate binding. For example, residues that can be altered to introduce desirable interactions with a substrate, intermediate, or product can be identified. Such a residue can be replaced with a residue that is complementary with a feature of the substrate, intermediate, or product, for example, with a charged residue (e.g., lysine, arginine, or histidine) that can electrostatically interact with an oppositely charged moiety on the substrate, intermediate, or product (e.g., a carboxylic acid group), a hydrophobic residue that can interact with a hydrophobic group on the substrate, intermediate, or product, or a residue that can hydrogen bond to the substrate, intermediate, or product (e.g., serine, threonine, histidine, asparagine, or glutamine). Residues that are undesirably close to the projected location of one or more atoms within the substrate, intermediate, or product can similarly be identified. Such a residue can, for example, be deleted or replaced with a residue having a smaller side chain, e.g., to accommodate a larger substrate or product; for example, many residues can be conveniently replaced with a residue having similar characteristics but a shorter amino acid side chain, or, e.g., with alanine. Residues identified as targets for mutagenesis can, for example, be mutated to predetermined residues, or mutagenesis of the target residues can be essentially random, followed by selection of proteins with desired substrate preference, catalytic activity, or the like from a library of mutant proteins.

As one example of such structure-based design, examination of a TAL-coumarate lyase-product complex structure revealed that a histidine residue of the TAL (His 89 of Rhodobacter sphaeroides TAL) hydrogen bonds with the p-hydroxyl group of the coumarate product. Mutation of this histidine residue to a phenylalanine altered substrate preference of the mutant lyase from Tyr to Phe, as described in greater detail in the Examples sections below. Thus, based on information from the TAL-product complex structure, the substrate preference of an amino acid ammonia lyase can be altered by modifying the lyase. For example, the substrate preference of a TAL is optionally switched from Tyr to Phe by mutating residue 89 to Phe (and optionally also mutating residue 90 to Leu) or from Tyr to His by mutating residue 89 to Ser (and optionally also mutating residue 90 to H is); the substrate preference of a PAL is optionally switched from Phe to Tyr by mutating residue 89 to His (and optionally also mutating residue 90 to Leu) or from Phe to His by mutating residue 89 to Ser (and optionally also mutating residue 90 to H is); and the substrate preference of a HAL is optionally switched from His to Tyr by mutating residue 89 to His (and optionally also mutating residue 90 to Leu) or from His to Phe by mutating residue 89 to Phe (and optionally also mutating residue 90 to Leu); where residues are numbered corresponding to those of Rhodobacter sphaeroides TAL.

Information from an amino acid ammonia lyase-product complex structure or a TAL structure can similarly be used to predict which residues or regions of an amino acid ammonia lyase can be mutated to alter specificity of the lyase from, e.g., Tyr, Phe, or H is, to a rare or non-standard amino acid such as L-tryptophan, L-DOPA, or even to an unnatural amino acid (for example, other hydroxylated phenylalanines, halogenated phenylalanines, pyridinylalanines, pyrimidinylalanines, and naphthyl-alanines). For example, residues lining the binding pocket can be mutated as described above, in particular, His89, Leu90, Leu153, and Val409 (example numbering is with respect to RsTAL).

Similarly, information from an amino acid ammonia lyase-product complex structure or a TAL structure can be used to predict which residues or regions of an amino acid ammonia lyase can be mutated to alter catalytic activity of the enzyme. By altering interactions between a substrate, intermediate, or product in the enzyme, for example, one or more mutations can transform an amino acid ammonia lyase to an aminomutase. (As described above, the MIO-dependent aminomutases are closely related structurally and mechanistically to the aromatic amino acid ammonia lyases and convert α-amino acid substrates to β-amino acid products.) Useful targets for mutation include Gly348 and Gly349 (RsTAL residue numbering); in general, residues conferring aminomutase activity are desirably modified.

The methods of modifying enzymes based on information from the crystal structure of an amino acid ammonia lyase-product complex or a TAL can be extended to enzymes other than amino acid ammonia lyases. Essentially any enzyme or other protein with sufficient homology to a lyase can be modified based on the structure of that lyase or its complex. Thus, in one aspect, an enzyme such as an aminomutase is modified based on information derived from an amino acid ammonia lyase-product complex structure or a TAL structure.

Systems related to the methods form another feature of the invention. Systems of the invention can include an information storage module (e.g., disk drive or optical disk), typically an information storage module comprising an information set derived from a crystal structure of an amino acid ammonia lyase enzyme bound to a product or from a crystal structure of a tyrosine ammonia lyase type enzyme. The system optionally also includes any of the various crystallographic or modeling software described above, e.g., implemented in a computer system. Systems also typically include one or more databases of crystallographic information. Systems also optionally include a user input device (e.g., keyboard or mouse), a user viewable display, etc. Optionally, the system can include one or more modules that assist in gathering crystallographic information, e.g., any of those noted above.

### Determining Kinetic Parameters

The recombinant amino acid ammonia lyase enzymes of the invention can be screened or otherwise tested to determine whether the recombinant enzyme displays an altered substrate preference as compared, e.g., to the corresponding lyase enzyme from which the recombinant enzyme was derived. Similarly, other modified enzymes of the invention can be screened or otherwise tested to determine whether the enzyme displays a modified activity for or with a given substrate as compared, e.g., to the corresponding wild-type enzyme from which the modified enzyme was derived.

For example, to determine the substrate preference of a recombinant amino acid ammonia lyase, k_{cat}, Kₘ, Vₘₐₓ, Vₘₐₓ/Kₘ, and/or k_{cat}/Kₘ of the recombinant amino acid ammonia lyase for a first substrate can be determined. Further, k_{cat}, Kₘ, Vₘₐₓ, Vₘₐₓ/Kₘ, and/or k_{cat}/Kₘ of the recombinant amino acid ammonia lyase for a second substrate can also be determined. Comparison of the kinetic parameters of the recombinant enzyme for the two substrates can indicate which substrate the enzyme prefers. For example, a preferred substrate has a lower Kₘ and/or a higher k_{cat}/Kₘ than a less preferred substrate. It is worth noting that k_{cat} and Kₘ are typically not determinable for a substrate that the enzyme does not significantly utilize.

As is well-known in the art, for enzymes obeying simple Michaelis-Menten kinetics, kinetic parameters are readily derived from rates of catalysis measured at different substrate concentrations. The Michaelis-Menten equation, V=Vₘₐₓ[S]([S]+Kₘ)⁻¹, relates the concentration of uncombined substrate ([S], approximated by the total substrate concentration), the maximal rate (Vₘₐₓ, attained when the enzyme is saturated with substrate), and the Michaelis constant (Kₘ, equal to the substrate concentration at which the reaction rate is half of its maximal value), to the reaction rate (V).

For many enzymes, Kₘ is equal to the dissociation constant of the enzyme-substrate complex and is thus a measure of the strength of the enzyme-substrate complex. For such an enzyme, in a comparison of Kₘs, a lower Kₘ represents a complex with stronger binding, while a higher Km represents a complex with weaker binding.

The ratio k_{cat}/Kₘ, sometimes called the specificity constant, represents the apparent rate constant for combination of substrate with free enzyme. The larger the specificity constant, the more efficient the enzyme is in binding the substrate and converting it to product.

The k_{cat} (also called the turnover number of the enzyme) can be determined if the total enzyme concentration ([E_{T}], i.e., the concentration of active sites) is known, since Vₘₐₓ=k_{cat}[E_{T}]. For situations in which the total enzyme concentration is difficult to measure, the ratio Vₘₐₓ/Kₘ is optionally used instead as a measure of efficiency. Kₘ and Vₘₐₓ can be determined, for example, from a Hanes plot, from a Lineweaver-Burk plot of 1/V against 1/[S], where the y intercept represents 1/Vₘₐₓ, the x intercept −1/Kₘ, and the slope Kₘ/Vₘₐₓ, or from an Eadie-Hofstee plot of V against V/[S], where the y intercept represents Vₘₐₓ, the x intercept Vₘₐₓ/Kₘ, and the slope —Kₘ. Software packages such as KinetAsyst™ or Enzfit (Biosoft, Cambridge, UK) can facilitate the determination of kinetic parameters from catalytic rate data.

Specific examples of determination of kinetic parameters and substrate preference for various wild type and mutant amino acid ammonia lyases are described in the Examples sections below. The activity of the ammonia-lyase enzymes can be readily assayed spectrophotometrically, by monitoring the absorbance change due to formation of an aryl-acrylic acid product (see, for example, J. A. Kyndt, T. E. Meyer, M. A. Cusanovich and J. J. Van Beeumen 2002, FEBS Lett. 512, 240-4).

For a more thorough discussion of enzyme kinetics, see, e.g., Berg, Tymoczko, and Stryer (2002) Biochemistry, Fifth Edition, W. H. Freeman; Creighton (1984) Proteins: Structures and Molecular Principles, W. H. Freeman; and Fersht (1985) Enzyme Structure and Mechanism, Second Edition, W. H. Freeman.

Screening Enzymes

Screening or other protocols can be used to determine whether an enzyme (e.g., an amino acid ammonia lyase) displays a desired activity for a given substrate. For example, k_{cat}, Kₘ, Vₘₐₓ, Vₘₐₓ/Kₘ, or k_{cat}/Kₘ of a mutant amino acid ammonia lyase for the substrate can be determined as discussed above. Further, the k_{cat}, Kₘ, Vₘₐₓ, Vₘₐₓ/Kₘ, or k_{cat}/Kₘ can be compared to that for a different substrate or to that of a parental enzyme for the substrate.

In one desirable aspect, a library of amino acid ammonia lyase polypeptides can be made and screened for these properties. For example, a plurality of members of the library can be made to collectively comprise a plurality of mutations of one or more amino acids in at least one region of the polypeptides, the region corresponding to an active site of an amino acid ammonia lyase enzyme, and the library can then be screened for the properties of interest. In general, the library can be screened to identify at least one member comprising a modified activity of interest (e.g., altered substrate preference, altered catalytic activity, or the like).

Libraries of amino acid ammonia lyase polypeptides can be either physical or logical in nature. Moreover, any of a wide variety of library formats can be used. For example, polypeptides can be fixed to solid surfaces in arrays of polypeptides. Similarly, liquid phase arrays of polypeptides (e.g., in microwell plates) can be constructed for convenient high-throughput fluid manipulations of solutions comprising polypeptides. Liquid, emulsion, or gel-phase libraries of cells that express amino acid ammonia lyase polypeptides can also be constructed, e.g., in microwell plates, or on agar plates. Phage display libraries of amino acid ammonia lyases or amino acid ammonia lyase polypeptides (e.g., including the active site region) can be produced. Instructions in making and using libraries can be found, e.g., in Sambrook, Ausubel and Berger, referenced herein.

For the generation of libraries involving fluid transfer to or from microtiter plates, a fluid handling station is optionally used. Several “off the shelf” fluid handling stations for performing such transfers are commercially available, including e.g., the Zymate systems from Caliper Life Sciences (Hopkinton, Mass.) and other stations which utilize automatic pipettors, e.g., in conjunction with the robotics for plate movement (e.g., the ORCA® robot, which is used in a variety of laboratory systems available, e.g., from Beckman Coulter, Inc. (Fullerton, Calif.).

In an alternate embodiment, fluid handling is performed in microchips, e.g., involving transfer of materials from microwell plates or other wells through microchannels on the chips to destination sites (microchannel regions, wells, chambers or the like). Commercially available microfluidic systems include those from Hewlett-Packard/Agilent Technologies (e.g., the HP2100 bioanalyzer) and the Caliper High Throughput Screening System. The Caliper High Throughput Screening System provides one example interface between standard microwell library formats and Labchip technologies. Furthermore, the patent and technical literature includes many examples of microfluidic systems which can interface directly with microwell plates for fluid handling.

Detecting Enzymes, Nucleic Acids and Phenylpropanoids

Expression of the recombinant amino acid ammonia lyase in a host cell and/or expression of additional enzymes in the host cell (e.g., enzymes for precursor synthesis and/or downstream enzymes that convert the product of the recombinant amino acid ammonia lyase into a final phenylpropanoid product) can be verified at the mRNA or protein level using techniques well known in the art. For example, expression of one or more enzyme can be detected by reverse transcription-polymerase chain reaction (RT-PCR) or northern analysis (for detection of mRNA) or by dot blots or Western analysis (for protein detection). See, e.g., Sambrook, Ausubel and Berger, all infra. Further details on protein (e.g., enzyme) and nucleic acid purification and detection are also found below.

The product of the recombinant amino acid ammonia lyase (in vivo or in vitro) can similarly be detected and/or identified using techniques well known in the art, as can any precursors synthesized in the host cell, intermediates between the product of the lyase and a final phenylpropanoid product, and/or the final phenylpropanoid product. Suitable techniques include, for example, high-performance liquid chromatography (HPLC), liquid chromatography-mass spectrometry (LC-MS), tandem mass spectrometry (MS/MS), and gas chromatography-mass spectrometry (GC-MS). See, e.g., Jiang et al., Hwang et al., and Mayer et al., all supra. The phenylpropanoid product is optionally purified, using techniques well known in the art.

### Applications for Recombinant Amino Acid Ammonia Lyases

The recombinant amino acid ammonia lyases of the invention have a variety of applications. For example, the recombinant amino acid ammonia lyases are useful for in vitro or in vivo engineering of phenylpropanoid synthetic pathways. As another example, recombinant amino acid ammonia lyases having PAL activity are candidates for enzyme substitution therapy for treatment of phenylketonuria.

With respect to human clinical conditions, TAL does not appear naturally to occur in humans. HAL (also referred to as histidase) is a naturally occurring enzyme in animals. Deficiency of HAL activity in humans is the cause of histidinemia, which results in elevated levels of histidine in the blood, urine, and cerebrospinal fluid. However, the consequences of histidinemia are relatively benign, except in rare cases that involve disorders of the central nervous system. For these cases, enzyme-replacement therapy with HAL could be useful. Other, non-clinical applications of the ammonia-lyase enzymes are discussed in other sections of this application.

Phenylpropanoid Pathway Engineering

Phenylpropanoids are a class of organic compounds (typically plant derived from natural sources) that are biosynthesized from the amino acid phenylalanine. In nature, they have a wide variety of functions, including defense against herbivores, microbial attack, or other sources of injury, as structural components of cell walls (e.g., lignins); as protection from ultraviolet light, as pigments, and as signaling molecules.

Typically, phenylalanine is converted to cinnamic acid by the action of a phenylalanine ammonia lysase (PAL). A series of enzymatic hydroxylations and methylations leads to coumaric acid, caffeic acid, ferulic acid, 5-hydroxyferulic acid, and sinapic acid. Conversion of these acids to their corresponding esters produces some of the volatile components of herb and flower fragrances which serve many functions such as attracting pollinators. Ethyl cinnamate is a common example.

In addition, reduction of a carboxylic acid functional group in a cinnamic acid provides a corresponding aldehyde, such as cinnamaldehyde. Further reduction provides monolinguals including coumaryl alcohol, coniferyl alcohol, and sinapyl alcohol. The monolinguals are monomers that can be polymerized to generate various forms of lignin and suberin, which are used as a structural component of, e.g., plant cell walls. The phenylpropenes, including eugenol, chavicol, safrole and estragole, are also derived from the monolinguals. These compounds are the primary constituents of various essential oils.

Further, hydroxylation of cinnamic acid in the 2-position leads to coumarin, which can be further modified into hydroxylated derivatives such as umbelliferone. Additional elaboration provides the flavonoids, a diverse class of phytochemicals.

Accordingly, phenylpropanoids have a broad range of activities and uses, for example, as fragrances, cell wall constituents, flavors, and antibiotics. Phenylpropanoids are also desirable lead compounds, since in addition to antibiotic activity, phenylpropanoids have been determined to possess other desirable properties such as anti-inflammatory, antiallergenic, antioxidant, and anticancer activities. Pathway engineering for production of various phenylpropanoids, for example, novel phenylpropanoids or phenylpropanoids difficult to obtain in sufficient quantities from natural sources, is therefore of considerable interest and immediate commercial value.

The recombinant amino acid ammonia lyases described herein are optionally used to produce precursors for synthesis of such phenylpropanoids. For example, recombinant amino acid ammonia lyase enzymes with PAL or TAL activity can produce the phenylpropanoid precursors cinnamic acid and coumaric acid, respectively. Similarly, recombinant enzymes that act on rare, non-standard, or unnatural amino acids can produce other precursors useful for phenylpropanoid synthesis.

For phenylpropanoid pathway engineering, the recombinant amino acid ammonia lyase is typically expressed in a host cell, e.g., as described herein. The host cell is optionally one that does not naturally produce phenylpropanoids or that does not naturally express a PAL and/or TAL, such as many bacteria. Exemplary host cells also include amino acid ammonia lyase gene modified (or knockout) versions of natural hosts. Exemplary host cells include, but are not limited to, prokaryotic cells such as E. coli and other bacteria and eukaryotic cells such as yeast, plant, insect, amphibian, avian, and mammalian cells, including human cells. Bacteria with a higher or lower AT vs. GC content in their genomes relative to E. coli are optionally used as host cells, to optimize expression of similarly-biased genes; for example, S. coelicolor or S. lividans is optionally used for expression of GC-rich constructs (Anne and Van Mellaert (1993) “Streptomyces lividans as host for heterologous protein production” FEMS Microbiol Lett. 114(2):121-8), while Pseudomonas species are optionally used for expression of AT-rich constructs.

Where in vivo production of a phenylpropanoid product by the host cell is desired, the precursors required for phenylpropanoid synthesis (e.g., Tyr or Phe or other natural or unnatural amino acids) can be endogenous to the cell, such precursors can be provided exogenously and taken up by the cell, and/or biosynthetic pathway(s) to create the precursors in vivo can be generated or engineered into the host cell. For example, biosynthetic pathways for non-standard or unnatural amino acids are optionally generated in the host cell by adding new enzymes or translation machinery (e.g., the use of orthogonal tRNA or RS components for the incorporation of unnatural amino acids) or for modifying existing host cell biosynthetic pathways.

A host cell expressing a recombinant amino acid ammonia lyase of the invention for production of phenylpropanoids also optionally expresses one or more additional enzymes, for example, enzymes whose collective action converts a product of the recombinant amino acid ammonia lyase into a final phenylpropanoid product. Such downstream tailoring enzymes can perform hydroxylation, methylation, reduction, and/or similar steps as necessary to produce the desired final product. Any such downstream enzymes can be expressed endogenously and/or heterologously in the host cell. A large number of enzymes involved in various phenylpropanoid biosynthetic pathways in a number of different species have been identified and are known in the art, for example, 4-coumaroyl:coenzyme A ligase, cinnamate 4-hydroxylase, chalcone synthase, chalcone isomerase, chalcone reductase, dihydroflavonol 4-reductase, 7, 29-dihydroxy, 49-methoxyisoflavanol dehydratase, flavanone 3-hydroxylase, flavone synthase (FSI and FSII), flavonoid 39 hydroxylase, flavonoid 3959 hydroxylase, isoflavone O-methyltransferase, isoflavone reductase, isoflavone 29-hydroxylase, isoflavone synthase, leucoanthocyanidin dioxygenase, leucoanthocyanidin reductase, O-methyltransferase, rhamnosyl transferase, stilbene synthase, UDPG flavonoid glucosyl transferase, and vestitone reductase, among many others. See, e.g., Winkel-Shirley (2001) “Flavonoid biosynthesis: A colorful model for genetics, biochemistry, cell biology, and biotechnology” Plant Physiology 126:485-493, Jiang et al. (2005) “Metabolic engineering of the phenylpropanoid pathway in Saccharomyces cerevisiae” Appl. Envir. Microbiol. 71:2962-2969, Hwang et al. (2003) “Production of plant-specific flavanones by Escherichia coli containing an artificial gene cluster” Appl. Environ. Microbiol. 69:2699-2706, Watts et al. (2004) “Exploring recombinant flavonoid biosynthesis in metabolically engineered Escherichia coli” Chembiochem 5:500-507, and Mayer et al. (2001) “Rerouting the plant phenylpropanoid pathway by expression of a novel bacterial enoyl-CoA hydratase/lyase enzyme function” Plant Cell 13:1669-1682.

Additional new enzymes expressed in the host cell (e.g., for precursor synthesis and/or downstream enzymes that convert the product of the recombinant amino acid ammonia lyase into a final phenylpropanoid product) are optionally naturally occurring enzymes, e.g., from other species, or artificially evolved enzymes. The genes for these enzymes can be introduced into a cell by transforming the cell with a plasmid comprising the genes and/or integrating the genes into the host's genome. The genes, when expressed in the cell, provide an enzymatic pathway to synthesize the desired phenylpropanoid compound. Examples of the types of enzymes that are optionally added are provided herein, and additional enzyme sequences can be found, e.g., in Genbank and in the literature.

Where artificially evolved enzymes are added into the cell, any of a variety of methods can be used for producing novel enzymes, e.g., for use in biosynthetic pathways or for evolution of existing pathways, in vitro or in vivo. Many available methods of evolving enzymes and other biosynthetic pathway components can be applied to the present invention to produce precursors or products (or, indeed, to evolve lyases or domains thereof to have new substrate specificities or other activities of interest). For example, DNA shuffling is optionally used to develop novel enzymes and/or pathways of such enzymes for the production of precursors or products, in vitro or in vivo. See, e.g., Stemmer (1994) “Rapid evolution of a protein in vitro by DNA shuffling” Nature 370(4):389-391; and, Stemmer, (1994) “DNA shuffling by random fragmentation and reassembly: In vitro recombination for molecular evolution” Proc. Natl. Acad. Sci. USA., 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution” Nature, 391(6664):288-291. New enzymes can also be generated using a DNA recombination procedure known as “incremental truncation for the creation of hybrid enzymes” (“ITCHY”), e.g., as described in Ostermeier et al. (1999) “A combinatorial approach to hybrid enzymes independent of DNA homology” Nature Biotech 17:1205. This approach can also be used to generate a library of enzyme or other pathway variants which can serve as substrates for one or more in vitro or in vivo recombination methods. See, also, Ostermeier et al. (1999) “Combinatorial Protein Engineering by Incremental Truncation” Proc. Natl. Acad. Sci. USA 96: 3562-67, and Ostermeier et al. (1999), “Incremental Truncation as a Strategy in the Engineering of Novel Biocatalysts” Biological and Medicinal Chemistry 7:2139-44. Another approach uses exponential ensemble mutagenesis to produce libraries of enzyme or other pathway variants that are, e.g., selected for an ability to catalyze a biosynthetic reaction relevant to producing a precursor or product. In this approach, small groups of residues in a sequence of interest are randomized in parallel to identify, at each altered position, amino acids which lead to functional proteins. Examples of such procedures, which can be adapted to the present invention to produce new enzymes for the production of precursors or phenylpropanoid products are found in Delegrave and Youvan (1993) Biotechnology Research 11:1548-1552. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more biosynthetic pathway function (e.g., for the production of precursors or products in vivo). See, e.g., Short “Non-Stochastic Generation of Genetic Vaccines and Enzymes” WO 00/46344.

Lyase or mutase enzymes of the invention, and/or related enzymes that typically act in concert with these enzymes, e.g., in biosynthetic pathways, can also be modified, e.g., at the relevant active site, to include any of a variety of unnatural amino acids. The incorporation of unnatural amino acids at the active site provides novel activities for the enzymes. A variety of unnatural amino acids, as well as methods of genetically encoding them into proteins, in vivo, using orthogonal tRNA-orthogonal aminoacyl synthetases are described in the literature. See, e.g., Wang and Schultz, “Expanding the Genetic Code,” Chem. Commun. (Camb.) 1:1-11 (2002); Wang and Schultz “Expanding the Genetic Code,” Angewandte Chemie Int. Ed., 44(1):34-66 (2005); Xie and Schultz, “An Expanding Genetic Code,” Methods 36(3):227-238 (2005); Xie and Schultz, “Adding Amino Acids to the Genetic Repertoire,” Curr. Opinion in Chemical Biology 9(6):548-554 (2005); Wang et al., “Expanding the Genetic Code,” Annu. Rev. Biophys. Biomol. Struct., 35:225-249 (2006; epub Jan. 13, 2006); and Xie and Schultz, “A chemical toolkit for proteins—an expanded genetic code,” Nat. Rev. Mol. Cell. Biol., 7(10):775-782 (2006; epub Aug. 23, 2006). For example, larger (than Trp) amino-acids that would block the active site and confer specificity toward non-aromatic amino-acid substrates may be useful. Mutant libraries of natural or unnatural amino acid containing enzymes, focused on the active site surface are useful for selection against particular substrates, e.g., as described herein.

In addition, serum half-life and other properties of enzymes can be modulated using well known methods, such as by the addition of PEG or other protective (e.g., saccharide) moieties to the enzymes. This can be done by standard chemical methods, or by encoding appropriate unnatural amino acids into the enzyme for reactive coupling with PEG or other protective moieties.

An alternative to such mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce a desired precursor or product (or intermediate thereof). For example, methods taught in the following publications can be applied to pathway design for the evolution of existing and/or new pathways in cells to produce precursors or products in vivo: Patnaik et al. (2002) “Genome shuffling of lactobacillus for improved acid tolerance” Nature Biotechnology 20(7):707-712; and Zhang et al. (2002) “Genome shuffling leads to rapid phenotypic improvement in bacteria” Nature 415:644-646.

Other techniques for organism and metabolic pathway engineering, e.g., for the production of desired compounds, are also available and can also be applied to the production of precursors or phenylpropanoid products. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry 41(20):6226-36; Selivonova et al. (2001) “Rapid Evolution of Novel Traits in Microorganisms” Applied and Environmental Microbiology 67:3645, and many others.

Regardless of the method used, typically, the precursor(s) produced with an engineered biosynthetic pathway of the invention is produced in a concentration sufficient for efficient phenylpropanoid biosynthesis, e.g., a natural cellular amount, but not to such a degree as to significantly affect the concentration of other cellular compounds or to exhaust cellular resources. Once a cell is engineered to produce enzymes desired for a specific pathway and a precursor is generated or provided, in vivo selections are optionally used to further optimize the production of the precursor for both phenylpropanoid synthesis and cell growth.

Treatment of Phenylketonuria and Other Disorders

The inherited metabolic disease phenylketonuria (PKU) is caused by mutation in the enzyme phenylalanine hydroxylase, which normally converts phenylalanine to tyrosine. In the absence of phenylalanine hydroxylase, excess phenylalanine from the diet cannot be eliminated, and phenylalanine and its breakdown products from other routes accumulate in the body with neurotoxic effects. To prevent mental retardation, individuals with PKU currently have to maintain a rigid diet.

PALs have been investigated as an enzyme substitution therapy for treatment of individuals with PKU, since they can reduce levels of phenylalanine in the blood by converting it to the harmless products cinnamate and ammonia. The Rhodosporidium toruloides PAL can lower blood phenylalanine levels in mice. However, various factors such as susceptibility to proteolytic cleavage and immunogenicity have impeded clinical usage of this PAL. See, e.g., Wang et al. (2005) “Structure-based chemical modification strategy for enzyme replacement treatment of phenylketonuria. Mol Genet Metab 86:134-40, Sarkissian and Gamez (2005) “Phenylalanine ammonia lyase, enzyme substitution therapy for phenylketonuria, where are we now?” Mol Genet Metab 86 Suppl 1:S22-6, Sarkissian et al. (1999) “A different approach to treatment of phenylketonuria: phenylalanine degradation with recombinant phenylalanine ammonia lyase” Proc Natl Acad Sci USA 96:2339-44, and Ikeda et al. (2005) “Phenylalanine ammonia-lyase modified with polyethylene glycol: potential therapeutic agent for phenylketonuria” Amino Acids 29:283-7.

In general, prokaryotic PALs are smaller than their eukaryotic counterparts, suggesting that prokaryotic PALs may have advantages in terms of production, administration, and stability, for example. However, as noted above, relatively few prokaryotic PALs have been identified.

The methods and recombinant amino acid ammonia lyases of the invention can circumvent these difficulties by providing new enzymes with PAL activity. For example, a TAL or, particularly, a HAL (which tend to be smaller and which are widespread in nature) with desirable properties such as stability and/or high turnover can be modified as described herein to switch its substrate preference from tyrosine or histidine to phenylalanine, providing a useful PAL for enzyme replacement therapy. Typical methods for reducing the immunogenicity of a therapeutic protein include chemical addition of a modifying group as a means of masking antigenic sites, or site-directed mutagenesis as a means of removing predicted protein epitopes.

Similarly, amino acid ammonia lyases, including the recombinant amino acid ammonia lyases of the invention, may be useful for treatment of other disorders. For example, the R. sphaeroides TAL was demonstrated to convert L-DOPA to caffeic acid. The R. sphaeroides TAL or a recombinant amino acid ammonia lyase with similar activity could thus be useful in enzyme substitution therapy for conditions in which excessive levels of dopamine (for which L-DOPA is a precursor) are present, such as schizophrenia and Tourette's syndrome. Early studies indicated the possible carcinogenicity of high doses of caffeic acid, but more recently, caffeic acid has been shown to have beneficial effects, including anti-oxidant and anti-tumor activity. Another application of an L-DOPA ammonia-lyase is in lowering peripheral L-DOPA levels in the L-DOPA treatment of Parkinson's disease.

In general, for enzyme replacement/substitution therapies, the enzyme of the invention is introduced into contact with the patient using traditional administration methods (e.g., intravenous delivery). In a related approach, gene therapy can be used, in which the enzymes of the invention are encoded in an appropriate gene therapy vector, for expression of the vector at the target site.

Pharmaceutical Compositions

Enzymes (or modulators thereof, e.g., antibodies) and/or gene therapy vectors of the invention can be formulated into pharmaceutical compositions. These compositions may comprise, in addition to one or more enzymes or vectors, an available pharmaceutically acceptable excipient, carrier, buffer, stabilizer or the like. Such materials should typically be non-toxic and should not interfere with the efficacy of the active ingredient. The precise nature of the carrier or other material depends on the route of administration, e.g., whether administration is via oral, rectal, intravenous, cutaneous, subcutaneous, nasal, intramuscular, intraperitoneal or other routes.

For example, pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or local injection, e.g., at the site of an affliction, the active ingredient will be in the form of a parenterally acceptable aqueous solution which has suitable pH, isotonicity and stability. Those of skill in the art are able to prepare suitable solutions using, for example, isotonic vehicles such as sodium chloride injection, Ringer's injection, lactated Ringer's injection, or the like. Preservatives, stabilizers, buffers, antioxidants and/or other additives are also optionally included, as required.

Whether it is an enzyme, antibody to the enzyme, modulator of the enzyme or gene therapy vector that encodes the enzyme that is to be given to an individual, administration is preferably in a “prophylactically effective amount” (e.g., enough to prevent or ameliorate the effects of a disease, e.g., PKU) or a “therapeutically effective amount” (prophylaxis optionally also can be considered therapy), this being an amount sufficient to show a benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found, e.g., in the current edition of Remington's e.g., Remington: The Science and Practice of Pharmacy, Twenty First Edition (2005).

The compositions may be administered alone or in combination with other treatments, either simultaneously or sequentially dependent upon the condition to be treated. Thus, in the treatment of PKU, the enzymes, etc., can be administered in combination with other available therapies, diets, etc.

### Generation of Expression Vectors and Transgenic Cells

The present invention also relates to host cells and organisms which comprise recombinant nucleic acids corresponding to mutant amino acid ammonia lyases and structurally related enzymes such as amino acid mutases. Additionally, the invention provides for the production of recombinant polypeptides that provide improved flux through various biosynthetic pathways, e.g., for the improved production of phenylpropanoids.

The production of flavonoids and other phenylpropanoids is currently limited by the low growth rates of plants. Therefore, the transfer of plant metabolic pathways into heterologous hosts such as bacteria or Saccharomyces cerevisiae is an attractive alternative source of phenylpropanoids. In addition, the overexpression of genes that drive phenylpropanoid production in plants is desirable to increase plant-based production of phenylpropanoids. As has already been discussed, the expression of the enzymes of the invention also have clinical and veterinary uses in animals and human patients.

General texts which describe molecular biological techniques for the cloning and manipulation of nucleic acids and production of encoded polypeptides include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through the current date) (“Ausubel”)). These texts describe mutagenesis, the use of expression vectors, promoters and many other relevant topics related to, e.g., the generation of clones that comprise nucleic acids of interest, e.g., amino acid ammonia lyase or mutase proteins and coding genes.

Cell culture media in general are set forth in the previous references and, additionally, in Atlas and Parks (eds) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (e.g., 1997 or later) also from Sigma-Aldrich, Inc (St Louis, Mo.) (“Sigma-PCCS”). The culture of animal cells is described, e.g., by Freshney (2000) Culture of Animal Cells: A Manual Of Basic Techniques John Wiley and Sons, N.Y.

Host cells (plants, mammals, bacteria, fungi or others) are genetically engineered (e.g., transduced, transfected, transformed, etc.) with the vectors of this invention (e.g., vectors, such as expression vectors which comprise an ORF derived from or related to a lyase or mutase protein, e.g., a HAL, PAL or TAL) which can be, for example, a cloning vector, a shuttle vector or an expression vector. Such vectors are, for example, in the form of a plasmid, a phagemid, an agrobacterium, a virus, a naked polynucleotide (linear or circular), or a conjugated polynucleotide. Vectors to be expressed in eukaryotes can first be introduced into bacteria, especially for the purpose of propagation, expansion and protein production (e.g., for making crystals, etc.).

The engineered host cells can be cultured in conventional nutrient media modified as appropriate for such activities as, for example, activating promoters or selecting transformants.

When plant cells are the target for engineering, the cells can optionally be cultured into transgenic plants. In addition to Sambrook, Berger and Ausubel, all infra, Plant regeneration from cultured protoplasts is described in Evans et al. (1983) “Protoplast Isolation and Culture,” Handbook of Plant Cell Cultures 1, 124-176 (MacMillan Publishing Co., New York; Davey (1983) “Recent Developments in the Culture and Regeneration of Plant Protoplasts,” Protoplasts, pp. 12-29, (Birkhauser, Basel); Dale (1983) “Protoplast Culture and Plant Regeneration of Cereals and Other Recalcitrant Crops,” Protoplasts pp. 31-41, (Birkhauser, Basel); Binding (1985) “Regeneration of Plants,” Plant Protoplasts, pp. 21-73, (CRC Press, Boca Raton, Fla.). Additional details regarding plant cell culture and regeneration include Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Plant Molecular Biology (1993) R. R. D. Croy, Ed. Bios Scientific Publishers, Oxford, U.K. ISBN 0 12 198370 6.

It is not intended that plant transformation and expression of polypeptides that provide phenylpropanoid synthesis, as provided by the present invention, be limited to any particular plant species. Indeed, it is contemplated that amino acid ammonia lyase proteins can provide for phenylpropanoid metabolism engineering when transformed and expressed in any agronomically and horticulturally important species. Such species include dicots, e.g., of the families: Leguminosae (including pea, beans, lentil, peanut, yam bean, cowpeas, velvet beans, soybean, clover, alfalfa, lupine, vetch, lotus, sweet clover, wisteria, and sweetpea); and, Compositae (the largest family of vascular plants, including at least 1,000 genera, including important commercial crops such as sunflower), as well as monocots, such as from the family Graminae. Plants of the Rosaciae are also preferred targets. Additionally, preferred targets for modification with the nucleic acids of the invention, as well as those specified above, include plants from the genera: Agrostis, Allium, Anirrhinum, Apium, Arachis, Asparagus, Atropa, Avena, Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum, Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza, Panicum, Pelargonium, Pennisetum, Petunia, Pisum, Phaseolus, Phleum, Poa, Prunus, Ranunculus, Raphanus, Ribes, Ricinus, Rubus, Saccharum, Salpiglossis, Secale, Senecio, Setaria, Sinapis, Solanum, Sorghum, Stenotaphrum, Theobroma, Trifolium, Trigonella, Triticum, Vicia, Vigna, Vitis, Zea, the Olyreae, and the Pharoideae, and many others. Common crop plants which are targets of the present invention include: Arabidopsis thalina, Brassica naupus, Brassica juncea, Zea mays, soybean, sunflower, safflower, rapeseed, tobacco, canola, peas, beans, lentils, peanuts, yam beans, cowpeas, velvet beans, clover, alfalfa, lupine, vetch, sweet clover, sweetpea, field pea, fava bean, broccoli, Brussels sprouts, cabbage, cauliflower, kale, kohlrabi, celery, lettuce, carrot, onion, olive, pepper, potato, eggplant and tomato.

In addition, transgenic animals can also be made recombinant for a given lyase or mutase polypeptide, or a modified form thereof, thereby changing metabolism of one or more metabolite in the animal.

Xenopus and insect cells are useful targets for modification, due to the ease with which such cells can be grown, studied and manipulated. Human and veterinary patients can also be treated with gene therapy, e.g., with nucleic acids the encode a lyase with specificity for phenylalanine (e.g., a PAL or a substrate-switched TAL that is kinetically faithful to phenylalanine), or with enzyme replacement therapy (ERT).

A transgenic animal (e.g., a non-human animal) of the invention is typically an animal that has had DNA encoding a relevant enzyme of the invention introduced into one or more of its cells artificially. This is most commonly done in one of two ways. First, DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing heterologous DNA into embryonic stem (ES) cells and selecting for cells in which the heterologous DNA has undergone homologous recombination with homologous sequences of the cellular genome., Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted insertion of DNA is to make knock-out or transgenic mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

As applied to the present invention, endogenous genes relating to phenypropanoid synthetic pathways can be substituted for a amino acid ammonia lyase or mutase gene of the invention, and the effects of the introduced gene studied in the animal. In addition, the animal can be exposed to putative modulators of activity of the introduced gene (or encoded protein), and the effects on activity observed in the animal.

Transgenic animals capable of producing plant compounds in a tissue specific manner can be produced.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Isolating Pal, TAL and HAL Proteins from Natural or Recombinant Sources

Purification of amino acid ammonia lyase or mutase proteins can be accomplished using known techniques. Generally, cells expressing the proteins (naturally or by recombinant methods) are lysed, crude purification occurs to remove debris and some contaminating proteins, followed by chromatography to further purify the protein to the desired level of purity. Cells can be lysed by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during recombinant or natural synthesis, isolation or purification.

In general, amino acid ammonia lyase or mutase proteins can be purified, either partially (e.g., achieving a 5×, 10×, 100×, 500×, or 1000× or greater purification), or even substantially to homogeneity (e.g., where the protein is the main component of a solution, typically excluding the solvent (e.g., water, crystallization buffer, DMSO, or the like) and buffer components (e.g., salts and stabilizers) that the polypeptide is suspended in, e.g., if the polypeptide is in a liquid phase), according to standard procedures known to and used by those of skill in the art. Accordingly, polypeptides of the invention can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against amino acid ammonia lyase or mutase proteins are used as purification reagents, e.g., for affinity-based purification. Once purified, partially or to homogeneity, as desired, the polypeptides are optionally used e.g., as assay components, reagents, crystallization materials, or, e.g., as immunogens for antibody production.

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Biosenaration of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

Those of skill in the art will recognize that, after synthesis, expression and/or purification, proteins can possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art (see, the references above, and Debinski, et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

Amino acid ammonia lyase or mutase protein nucleic acids optionally comprise a coding sequence fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I., et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the sequence of the invention is useful to facilitate purification.

Specific example methods of purifying amino acid ammonia lyase proteins are described in the Examples sections below.

### Lyase Proteins and Genes

Amino acid ammonia lyase genes are modified (by switching substrate specificity) and expressed, e.g., to increase flux through phenylpropanoid and other synthetic pathways. For example, elevated expression of a TAL that has been substrate switched into acting as a PAL in a cell leads to increased production of trans-cinnamic acid, an intermediate in phenylpropanoid synthesis.

Amino acid ammonia lyase and other enzymes of interest herein include those proteins that share detectable homology to a known PAL, HAL or TAL enzymes, including a variety of PAL, HAL and TAL enzymes and substrate switched mutants, as well as amino mutase enzymes. Nucleic acids are homologous when they derive from a common ancestral nucleic acid, e.g., through natural evolution, or through artificial methods (mutation, gene synthesis, recombination, etc.). Homology between two or more proteins is usually inferred by consideration of sequence similarity of the proteins. Typically, protein sequences with as little as 25% identity, when aligned for maximum correspondence, are easily identified as being homologous. In addition, many amino acid substitutions are “conservative” having little effect on protein function. Thus, sequence alignment algorithms typically account for whether differences in sequence are conservative or non-conservative.

Thus, homology can be inferred by performing a sequence alignment, e.g., using BLASTN (for coding nucleic acids) or BLASTP (for polypeptides), e.g., with the programs set to default parameters. For example, in one embodiment, the protein is at least about 25%, at least about 50%, at least about 75%, at least about 80%, at least about 90% or at least about 95% identical to a known PAL, HAL or TAL, e.g., in the examples herein.

Homologous genes encode homologous proteins. Because of the degeneracy of the genetic code, the percentage of identity or similarity at which homology can be detected can be substantially lower than for the encoded polypeptides.

Sequence Comparison, Identity, and Homology

“Identity” or “similarity” in the context of two or more nucleic acid or polypeptide sequences, refers to the degree of sequence relatedness of the sequences. Typically, the sequences are aligned for maximum correspondence, and the percent identity or similarity is measured using a commonly available sequence comparison algorithm, e.g., as described below (other algorithms are available to persons of skill and can readily be substituted). Similarity can also be determined simply by visual inspection. Preferably, “identity” or “similarity” exists over a region of the sequences that is at least about 50 residues in length, more preferably over a region of at least about 100 residues, and most preferably the sequences are related over at least about 150 residues, or over the full length of the two sequences to be compared.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described, e.g., in Altschul et al., J. Mol. Biol. 215:403-410 (1990) and by Gish et al. (1993) “Identification of protein coding regions by database similarity search” Nature Genetics 3:266-72. Software for performing BLAST analyses is publicly available through the National Center for Biotechnology Information (www(dot)ncbi(dot)nlm(dot)nih(dot)gov/) and from Washington University (Saint Louis) at www(dot)blast(dot)wustl(dot)edu/. WU-blast 2.0 (latest release date Mar. 22, 2006) provides one convenient implementation of BLAST.

In general, this algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### Additional Details Regarding Sequence Variations

A number of particular amino acid ammonia lyase or mutase polypeptides and coding nucleic acids are described herein by sequence (See, e.g., the Examples sections below). These polypeptides and coding nucleic acids can be modified, e.g., by mutation as described herein, or simply by artificial synthesis of a desired variant. Several types of example variants are described below.

Silent Variations

Due to the degeneracy of the genetic code, any of a variety of nucleic acids sequences encoding polypeptides of the invention are optionally produced, some which can bear various levels of sequence identity to the amino acid ammonia lyase or mutase protein nucleic acids in the Examples below. The following provides a typical codon table specifying the genetic code, found in many biology and biochemistry texts.

TABLE A
Codon Table

| Amino acids | | | Codon | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

The codon table shows that many amino acids are encoded by more than one codon. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Such “silent variations” are one species of “conservatively modified variations”, discussed below. One of skill will recognize that each codon in a nucleic acid (except ATG, which is ordinarily the only codon for methionine) can be modified by standard techniques to encode a functionally identical polypeptide. Accordingly, each silent variation of a nucleic acid which encodes a polypeptide is implicit in any described sequence. The invention, therefore, explicitly provides each and every possible variation of a nucleic acid sequence encoding a polypeptide of the invention that could be made by selecting combinations based on possible codon choices. These combinations are made in accordance with the standard triplet genetic code (e.g., as set forth in Table A, or as is commonly available in the art) as applied to the nucleic acid sequence encoding a polypeptide of the invention. All such variations of every nucleic acid herein are specifically provided and described by consideration of the sequence in combination with the genetic code. One of skill is fully able to make these silent substitutions using the methods herein.

Conservative Variations

“Conservatively modified variations” or, simply, “conservative variations” of a particular nucleic acid sequence or polypeptide are those which encode identical or essentially identical amino acid sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known in the art. Table B sets forth six groups which contain amino acids that are “conservative substitutions” for one another.

TABLE B
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Thus, “conservatively substituted variations” of a listed polypeptide sequence of the present invention include substitutions of a small percentage, typically less than 5%, more typically less than 2% or 1%, of the amino acids of the polypeptide sequence, with a conservatively selected amino acid of the same conservative substitution group.

Finally, the addition or deletion of sequences which do not alter the encoded activity of a nucleic acid molecule, such as the addition or deletion of a non-functional sequence, is a conservative variation of the basic nucleic acid or polypeptide.

One of skill will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

Antibodies

In another aspect, antibodies to amino acid ammonia lyase or mutase polypeptides (e.g., type-switched enzymes) can be generated using methods that are well known. The antibodies can be utilized for detecting and/or purifying polypeptides e.g., in situ to monitor localization of the polypeptide, or simply for polypeptide detection in a biological sample of interest. Antibodies can optionally discriminate amino acid ammonia lyase or mutase polypeptide homologs (e.g., mutant type switched enzymes from native enzymes). Antibodies can also, in some cases, be used to modulate (e.g., block) function of amino acid ammonia lyase or mutase proteins, in vivo, in situ or in vitro (e.g., by binding to the active site on the protein).

As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

For the production of antibodies to a polypeptide encoded by one of the disclosed sequences or conservative variant or fragment thereof, various host animals may be immunized by injection with the polypeptide, or a portion thereof. Such host animals may include, but are not limited to, rabbits, mice and rats, to name but a few. Various adjuvants may be used to enhance the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al., Immunology Today 4:72, 1983; Cole et al., Proc. Nat'l. Acad. Sci. USA 80:2026-2030, 1983), and the EBV-hybridoma technique (Cole et al., Monoclonal Antibodies and Cancer Therapy, Alan R. Liss, Inc., pp. 77-96, 1985). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Nat'l. Acad. Sci. USA 81:6851-6855, 1984; Neuberger et al., Nature 312:604-608, 1984; Takeda et al., Nature 314:452-454, 1985) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science 242:423-426, 1988; Huston et al., Proc. Nat'l. Acad. Sci. USA 85:5879-5883, 1988; and Ward et al., Nature 334:544-546, 1989) can be adapted to produce differentially expressed gene-single chain antibodies. Single chain antibodies are formed by linking the heavy and light chain fragments of the Fv region via an amino acid bridge, resulting in a single-chain polypeptide.

In one aspect, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science 246:1275-1281, 1989) to allow rapid and easy identification of monoclonal Fab fragments with the desired specificity.

The protocols for detecting and measuring the expression of the described polypeptides herein, using the above mentioned antibodies, are well known in the art. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

One method, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically.

Alternately, fluorescent compounds, such as fluorescein and rhodamine, can be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Structural Determinants and Modulation of Substrate Specificity in Phenylalanine-Tyrosine Ammonia Lyases

The following sets forth a series of experiments that elucidate the structures of aromatic amino acid lyase-product complexes, identify residues that serve as key specificity determinants in the aromatic amino acid lyases, and demonstrate modification of specificity of an exemplary amino acid lyase from Tyr to Phe. Additional details may be found in Louie et al. “Structural Determinants and Modulation of Substrate Specificity in Phenyalanine-Tyrosine Ammonia-Lyases” Chemistry and Biology 13, 1327-1338 (2006), incorporated herein by reference in its entirety.

Aromatic amino acid ammonia lyases catalyze the deamination of L-His, L-Phe and L-Tyr, yielding ammonia plus aryl acids bearing an α,β-unsaturated, propenoic acid moiety. We describe herein high-resolution crystallographic analyses of unliganded Rhodobacter sphaeroides (Rs) Tyrosine Ammonia Lyase (TAL) and of RsTAL bound to the reaction products p-coumarate and caffeate. His 89 of RsTAL forms a hydrogen bond with the p-hydroxyl moieties of coumarate and caffeate. This residue is conserved in TALs but is replaced by other amino acids in Phenylalanine Ammonia Lyases (PALs) and Histidine Ammonia Lyases (HALs), and is therefore indicated to be important in discriminating between aromatic amino acid substrates. Replacement of His 89 by Phe, a characteristic residue of PALs, yields a mutant RsTAL with a marked switch in kinetic preference from L-Tyr to L-Phe. Additional structures, of the H89F mutant in complex with the PAL reaction product, cinnamate, or the PAL-specific inhibitor, 2-aminoindan-2-phosphonate (AIP), support the role of position 89 as a key specificity determinant in the larger family of aromatic amino acid ammonia lyases and the related aminomutases responsible for β-amino acid biosynthesis.

X-Ray Crystal Structure of R. sphaeroides TAL

The monoclinic crystal-form (space group P2₁) of RsTAL contains two complete homotetramers of the enzyme per asymmetric unit. The initial structure solution was obtained by molecular replacement, with a search model derived from a tetramer of P. putida HAL (Protein Data Bank (PDB) entry 1 GKM, available at www (dot) pdb (dot) org). The rotation function analysis identified two orientations for the tetramer, with peak heights of 13.4 and 12.5σ; translation-function searches then correctly positioned the two oriented tetramers, with an R-factor/correlation coefficient of 0.524/0.146 for the first tetramer, and subsequently, 0.514/0.182 for the second tetramer. The atomic model of RsTAL was refined to 1.58 Å resolution, resulting in the crystallographic statistics shown in Table 1. The two tetramers of RsTAL are nearly identical in structure, as the root-mean-squared positional deviation (rmsd) between equivalent backbone atoms is only 0.14 Å. The eight distinct monomers also exhibit nearly identical backbone conformations; for pair wise comparisons between individual monomers, the rmsd values range from 0.12 to 0.14 Å. Consistent with the extensive crystal packing (the solvent content is ˜40%), only residues 1 to 7 and the C-terminal residue 523 of each of the eight RsTAL monomers are poorly ordered in electron-density maps.

TABLE 1
Summary of data collection and refinement statistics.

| | RsTAL | RsTAL- | RsTAL- | H89F TAL- | H89F TAL- | H89F TAL- |
|---|---|---|---|---|---|---|
| | unliganded | coumarate | caffeate | cinnamate | coumarate | AIP |
| Space group | P2₁ | | | | | |
| Unit-cell parameters | | | | | | |
| a (Å) | 87.9 | 87.4 | 87.6 | 87.6 | 87.6 | 87.6 |
| b (Å) | 155.6 | 154.8 | 155.0 | 155.0 | 155.0 | 154.8 |
| c (Å) | 164.4 | 164.1 | 164.2 | 164.0 | 164.1 | 164.5 |
| β (°) | 94.1 | 94.1 | 94.1 | 94.2 | 94.1 | 94.2 |
| Monomers per | 8 | | | | | |
| asymmetric unit | | | | | | |
| Resolution range* | 100-1.50 | 100-1.58 | 100-1.90 | 100-1.90 | 100-2.00 | 100-1.75 |
| (Å) | (1.58-1.50) | (1.68-1.58) | (2.00-1.90) | (2.00-1.90) | (2.10-2.00) | (1.84-1.75) |
| Number of | 3870665 | 1901422 | 1195768 | 1133848 | 891554 | 1396599 |
| reflections measured | | | | | | |
| Merging R-factor* | 0.085 (0.618) | 0.068 (0.570) | 0.112 (0.546) | 0.135 (0.511) | 0.139 (0.503) | 0.089 (0.487) |
| Mean (I/σ I)* | 12.2 (2.3) | 15.6 (2.0) | 8.5 (2.4) | 7.5 (2.2) | 7.5 (2.5) | 9.3 (1.9) |
| Completeness* | 0.933 (0.859) | 0.873 (0.494) | 0.989 (0.975) | 0.992 (0.973) | 0.962 (0.959) | 0.964 (0.822) |
| Redundancy* | 5.9 (3.6) | 3.7 (2.7) | 3.5 (3.4) | 3.3 (2.6) | 3.2 (2.7) | 3.3 (2.2) |
| Number of | 644928 | 509395 | 338602 | 337722 | 282486 | 420387 |
| reflections used | | | | | | |
| R-factor* | 0.176 (0.254) | 0.192 (0.298) | 0.175 (0.253) | 0.178 (0.267) | 0.173 (0.247) | 0.189 (0.293) |
| Free R-factor* | 0.191 (0.276) | 0.208 (0.319) | 0.198 (0.283) | 0.200 (0.289) | 0.201 (0.281) | 0.210 (0.312) |
| Number of amino- | 4130 | 4130 | 4130 | 4130 | 4130 | 4130 |
| acid residues | | | | | | |
| Number of water | 3914 | 3481 | 3222 | 3302 | 3110 | 3091 |
| molecules | | | | | | |
| Residues with most | 93.4 | 93.3 | 92.6 | 92.6 | 92.3 | 92.9 |
| favorable | | | | | | |
| conformation (%) | | | | | | |

Merging R-factor = Σₕₖₗ Σᵢ | Iᵢ(hkl) − <I(hkl)> | /Σₕₖₗ Σᵢ Iᵢ(hkl)
*Values in parentheses describe the highest resolution shell.

Overall Structure of R. sphaeroides TAL

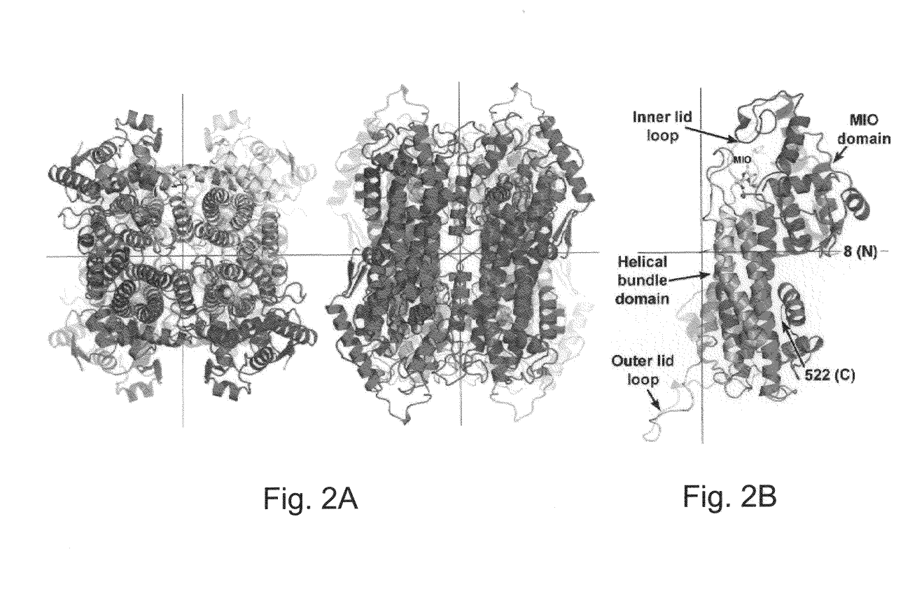

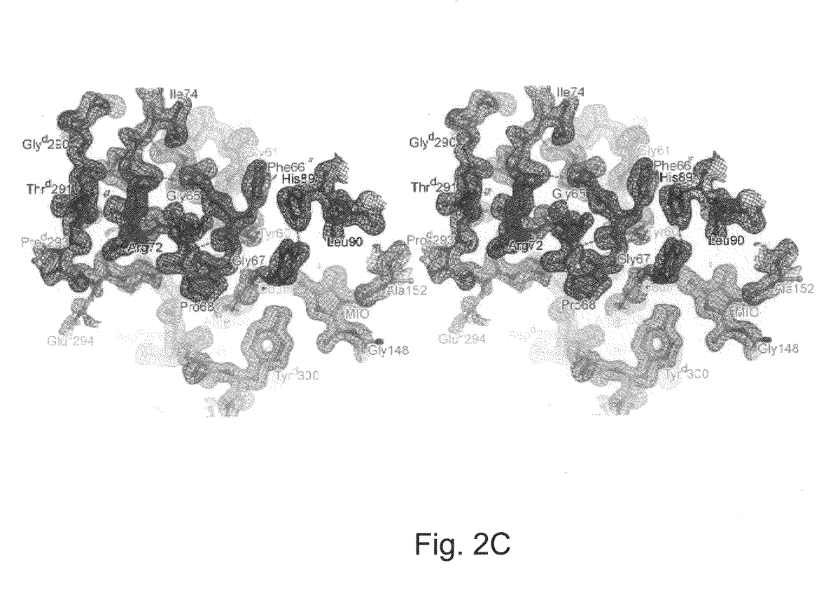

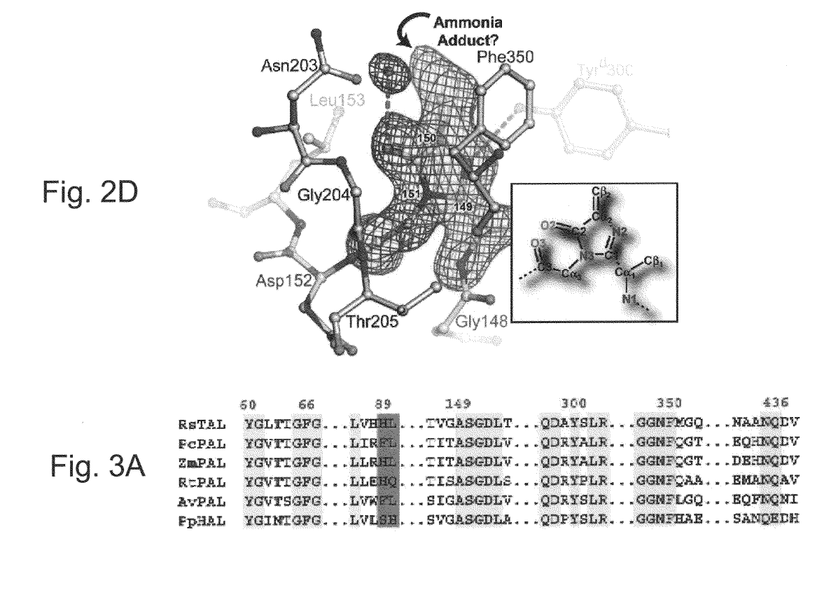

Both the tertiary and quaternary structures (FIGS. 2A and 2B) of RsTAL are highly similar to those described previously for related ammonia lyases, P. putida HAL (PDB entry 1 GKM) and PALs from parsley (P. crispum; PDB entry 1 W27) and yeast (R. toruloides; PDB entries 1T6P and 1Y2M). These proteins form homotetrameric oligomers with 222-point symmetry. The RsTAL homotetramer contains four active sites, with three distinct monomers participating in formation of each active-site cavity. Each monomer adopts a predominantly α-helical polypeptide-chain fold (FIG. 2B), which is organized around a central, up-down bundle of five α-helices. The flanking regions of these helices, together with the extended hairpin loop linking helices 4 and 5 of the helical bundle, are largely responsible for forming the monomer-monomer interfaces at the core of the tetramer. The N-terminal region of the polypeptide chain contributes to a domain that carries the MIO co-factor (FIG. 2B), and at the opposite end of the bundle, the C-terminal segment forms a peripheral α-helical layer. The C-terminal domain participates in additional inter-subunit contacts that stabilize the homotetramer, and in particular provides the outer-lid loop (FIGS. 2B and 2C, and discussion below), which caps the active-site cavity of an adjacent monomer. Like bacterial HAL, RsTAL lacks an additional domain that is inserted into the C-terminal domain of both yeast and parsley PAL. Without limitation to any particular mechanism, regulatory roles in shielding access to the active-site tunnel [1,8] or modulating the flexibility of an active-site lid loop [20] have been suggested as functional roles of this additional domain.

From a comparison of the superposed polypeptide-chain backbones of the known ammonia lyase structures, RsTAL differs from P. putida HAL by 2.3 Å rmsd for 485 equivalent residues, from parsley PAL by 2.4 Å rmsd for 480 equivalent residues and from yeast PAL by 2.3 Å rmsd for 475 equivalent residues. The slightly greater overall structural similarity of RsTAL with the bacterial HAL, in comparison to the eukaryotic PALs, is consistent with the higher overall sequence identity of RsTAL with the bacterial HALs and the absence of additional polypeptide segments (at the N-terminus and within the shielding domain) that occur in the eukaryotic PALs. These characteristics have led to suggestions that TAL and eukaryotic PAL diverged from bacterial HAL in separate evolutionary lineages [18].

Methylidene-Imidazolone Co-Factor of R. sphaeroides TAL

The MIO co-factor of RsTAL, formed by Ala 149, Ser 150 and Gly 151, resides in well-defined electron density (FIG. 2D). The imidazolone ring is stacked against the side chain of Phe 353. The MIO N2 atom, derived from Ser 150, accepts hydrogen bonds from the hydroxyl moiety of Tyr^{d}300 and the side-chain amide of Gln 436. Residue numbering refers to the polypeptide chain of a single monomer designated a; residues designated with superscripted b, c, or d are contributed by one of the dyad-related monomers of the homotetramer. The MIO keto group oxygen, 02 is directed into a pocket lined by the backbone amides of Leu 153 and Gly 204 (the oxyanion hole, see below) but does not make any direct contacts with protein residues and instead forms a hydrogen bond with a well-ordered water molecule (FIG. 2D).

In the crystal structure, the MIO co-factor appears to carry an adduct attached to the electrophilic methylidene carbon Cβ2 (FIG. 2D). This posited adduct is consistent with MIO derivatization by ammonia derived from ammonium acetate used for crystallization and present at 0.3 M. The presence of a nucleophilic adduct is supported by the planar sp² configuration of the MIO N3 atom, associated with aromaticity of the imidazolone ring. Similarly, crystal structures of other ammonia lyases bearing a covalent adduct indicate an sp² hybridization of N3 [13, 18, 19, 21]. In contrast, in structures of both PAL [17] and HAL [19] with an unmodified MIO co-factor, the N3 atom assumes an sp³ hybridization state, indicative of a non-aromatic imidazolone ring.

Binding of Coumarate and Caffeate Reaction Products to R. sphaeroides TAL

As noted above, we also solved the structures of TAL complexed with the products of the TAL-catalyzed reactions using L-Tyr and L-DOPA substrates, namely p-coumaric and caffeic acids, respectively. The stable binding of coumarate observed in RsTAL crystals is consistent with reported product inhibition of the ammonia-lyases [22]. However, attempts to complex L-Tyr or observe turnover of L-Tyr in RsTAL crystals were unsuccessful. The high concentration of ammonium ion in the crystallization medium and the attendant covalent modification of the MIO co-factor, as discussed above, likely prevent both L-Tyr binding and TAL activity in the crystals. Notably, HAL and PAL samples bearing modified co-factors have been shown to be catalytically inactive [18, 19]. In crystal soaking experiments of RsTAL with the PAL product cinnamic acid or the PAL-specific inhibitor, 2-aminoindan-2-phosphonic acid (AIP) [23], no significant binding was observed, in accord with the poor turnover of L-Phe by RsTAL and the weak inhibition of RsTAL activity by AIP (see discussion below and Table 2).

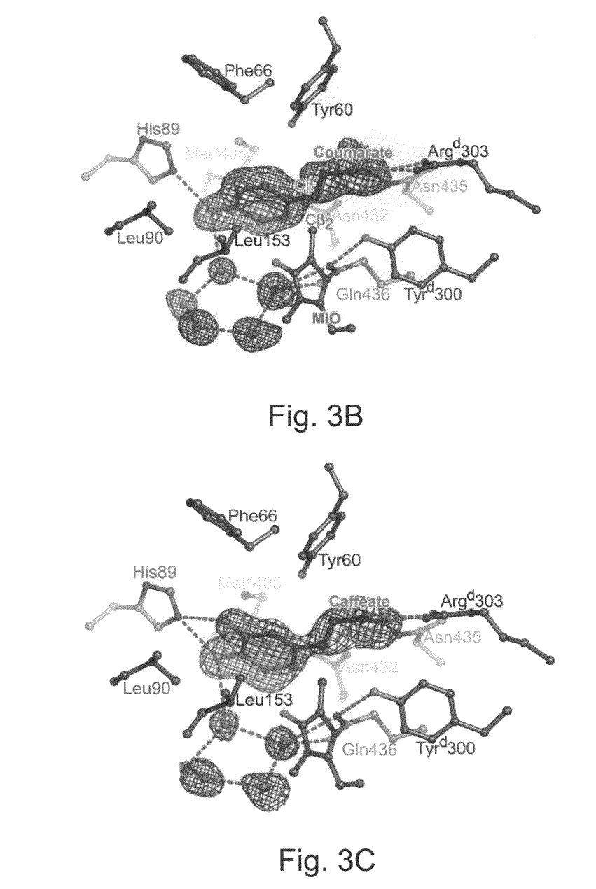

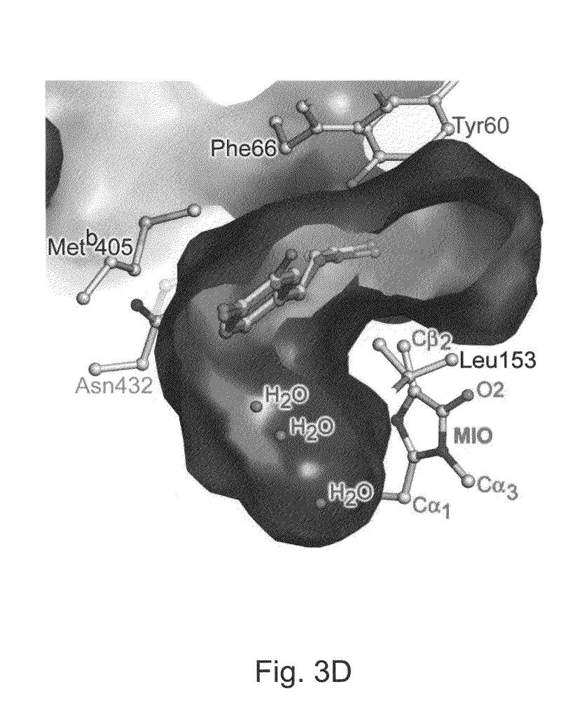

The RsTAL-coumarate co-crystal structure provides eight independent views (per asymmetric unit) of the enzyme-product complex. Notably, all of the protein residues that form interactions with coumarate are invariant in the TALs functionally characterized to date (FIG. 3A). The coumarate conformation and interactions with RsTAL are essentially identical in the eight copies of the complex in the asymmetric unit. In terms of the observed propenoate conformation, the alkene double bond is not conjugated with either the hydroxyphenyl ring (approximately 30° out of plane) or the carboxylate group (approximately 50° out of plane). Most notably, in all eight instances, the same (si) face of the alkene double bond is directed toward MIO. The hydroxyphenyl ring of coumarate is roughly orthogonal to the plane of the MIO co-factor. The closest approach to the electrophilic methylidene carbon of MIO −3.6 Å—is by the coumarate carbon atom equivalent to the Cβ atom in the L-Tyr substrate (FIG. 3B).

The carboxylate group of the propenoate moiety forms hydrogen bonds with the side-chain amide of Asn 435 and a salt bridge with the δ-guanido group of Arg^{d}303 from the dyad related polypeptide chain. The aliphatic portion of the propenoate segment packs against two residues from the inner lid-loop, Tyr 60 and Gly 67. The non-polar side-chains of Leu 90, Leu 153, and Met^{b}405, and the hydrophilic side-chains of Asn 432 and Gin 436 surround the phenyl ring of the bound coumarate. Finally, the p-hydroxyl group of the hydroxyphenyl ring forms hydrogen bonds with the side chain of His 89 and a water molecule that resides in a hydrogen-bonding network involving four other water molecules (FIG. 3B). Caffeic acid carries an additional meta-hydroxyl group on the phenyl ring, and in fact, the crystal structure of the caffeic acid complex with RsTAL demonstrates that this hydroxyl moiety sits in the space residing above the co-factor (FIG. 3C). Caffeic acid corresponds to the product of the deamination of the non-standard α-amino-acid L-DOPA (3,4-dihydroxy-L-Phe), and interestingly, RsTAL exhibits significant activity with L-DOPA.

Indeed, the L-shaped, active-site cavity is only partially filled by the coumarate molecule, and extends above the methylidene carbon of the MIO co-factor and into space occupied by the network of water molecules described above (FIGS. 3B and 3D). The excess space in the vicinity of the coumarate binding-site is available to phenyl rings with larger substituents as shown for the caffeic acid complex (FIGS. 3C and 3D). These results collectively indicate that RsTAL can optionally be deployed for in vivo generation of bioactive phenylpropanoids and that TALs can be rationally engineered to accept even more diverse amino-acid substrates, for example by the introduction of an additional active-site amino-acid residues capable of forming hydrogen bonds with polar groups on the targeted substrate, or by the creation of a larger binding pocket through active-site amino-acid replacements by glycine or serine residues.

Previous crystallographic analyses of PAL and HAL have yielded (with one exception) only unliganded enzyme structures, but these structures have served as a starting point for predictive modeling of substrate-, product-, and reaction-intermediate bound states of these enzymes. These modeling attempts have typically assumed attack on the MIO methylidene-carbon by either the α-amino group or the electron-rich aromatic ring of the substrate; and thus, substrate docking has targeted primarily a conjectured relative positioning of substrate and co-factor, as well as the formation of favorable substrate-protein interactions. Although some of these modeled complexes (in particular those described Ref. 1) agree roughly with the binding mode observed in the TAL-coumarate complex, in general, the ligand-enzyme interactions have not been accurately predicted at the atomic level. In addition, an apparently weakly bound cinnamate molecule described in a crystal structure of R. toruloides PAL [1,3] was modeled in a reverse orientation, with its phenyl and carboxylate groups approximately transposed relative to those of coumarate bound to TAL. Also, there is no apparent involvement of metal ions in the RsTAL-coumarate interaction, as proposed for HAL-substrate interactions [24].

The structures of the RsTAL-coumarate and RsTAL-caffeate complexes provide the first incontrovertible identification of the active-site residues that form the binding pocket for the product. Together with the pattern of amino acid conservation of the substrate recognition site (FIG. 3A) and the results of site-directed mutagenesis [24, 25] of the active-site residues in the aromatic amino acid ammonia lyase family, this structural information provides a rational basis for interpreting the roles that these residues play in determining substrate specificity and in catalyzing the enzyme reaction.

Tyr 60, Gly 67, Tyr 300, and Arg 303 (RsTAL numbering), which interact with the backbone atoms of the amino acid substrate, are highly conserved in HAL, PAL and TALs. Indeed, the salt bridge between the Arg 303 δ-guanido moiety and the substrate's α-carboxylate group served as an anchoring interaction in most of the earlier modeling attempts. Replacement of this conserved Arg with Ala in PAL [25] or Ile in HAL [24] caused large decreases in enzyme activity, whereas a Lys substitution in HAL minimally affected activity [24]. Phe substitution of the Tyr corresponding to RsTAL Tyr 300 in both HAL [24] and PAL [25] also resulted in significant losses of activity. Tyr 60, from the inner-lid loop (see below), likely resides near the Cβ atom of the amino acid substrates, and consistent with a postulated role as a general base for β-proton abstraction, substitution of this Tyr by Phe severely debilitated enzyme activity for both HAL [24] and PAL [25]. The residues that interact with the aromatic ring of the substrate are more variable among the ammonia lyases, but are more similar between the PAL and TAL families than between these two families and the HALs.

Notably, the His at the position corresponding to His 89 in RsTAL (or the adjacent position 90) is found in other functionally characterized bacterial TALs, as well as maize and yeast PALs, the latter of which possess significant TAL activity [2, 3]. In contrast, in PALs that are specific for L-Phe, a Phe occurs almost invariably at the position equivalent to RsTAL His 89, and Phe would support favorable non-polar interactions with the phenyl group of the L-Phe amino acid substrate. Furthermore, the pivotal two-residue His-Leu sequence (89 and 90 in RsTAL), which characterizes the TALs and is replaced by Phe-Leu in the PALs, is instead Ser-His in the HALs (FIG. 3A).

Structure-Based Switch of L-Tyr/L-Phe Substrate Preferences of R. sphaeroides TAL

Our structures clearly point to RsTAL His 89 as a key determinant of the substrate specificity of the aromatic amino acid ammonia lyases. We reasoned that replacement of His 89, which forms a favorable hydrogen bond with the side-chain hydroxyl group of L-Tyr, with Phe would result in considerable turnover of L-Phe. From an assessment of the kinetic properties of wild type (wt) and mutant RsTAL (Table 2), wild type RsTAL displays a marked kinetic preference for L-Tyr, as both the Kₘ (150-fold) and k_{cat}/Kₘ (53-fold) are substantially better for L-Tyr as compared to L-Phe. These values are very similar to the steady-state kinetic constants for the homologous TAL from Rhodobacter capsulatus [8].

In marked contrast, the engineered RsTAL variant with the single amino acid substitution H89F lacks activity with L-Tyr, and instead as predicted efficiently turns over L-Phe. With L-Phe, the H89F point mutant exhibits a 26-fold decrease in Kₘ and a 17-fold increase in k_{cat}/Kₘ in comparison to wild type RsTAL. Indeed, the catalytic efficiency of the PAL activity for the H89F mutant (k_{cat}/Kₘ 0.019 s⁻¹ μM⁻¹) is only slightly lower than that of TAL activity for wild type RsTAL (0.058 s⁻¹ μM⁻¹), and exceeds the catalytic efficiency of some native PALs (Table 2). In addition, the differing kinetic specificities of the wild type and mutant RsTALs are further substantiated by the relative susceptibilities to inhibition by AIP, a PAL-specific inhibitor [23]. For wild type RsTAL, activity with L-Tyr is unaffected by AIP, whereas activity with L-Phe is inhibited (Kᵢ=16.3 μM). In comparison, for the H89F mutant with L-Phe, the Kᵢ=0.60 μM and is 27-fold lower (greater inhibitory activity) than for wild type RsTAL.

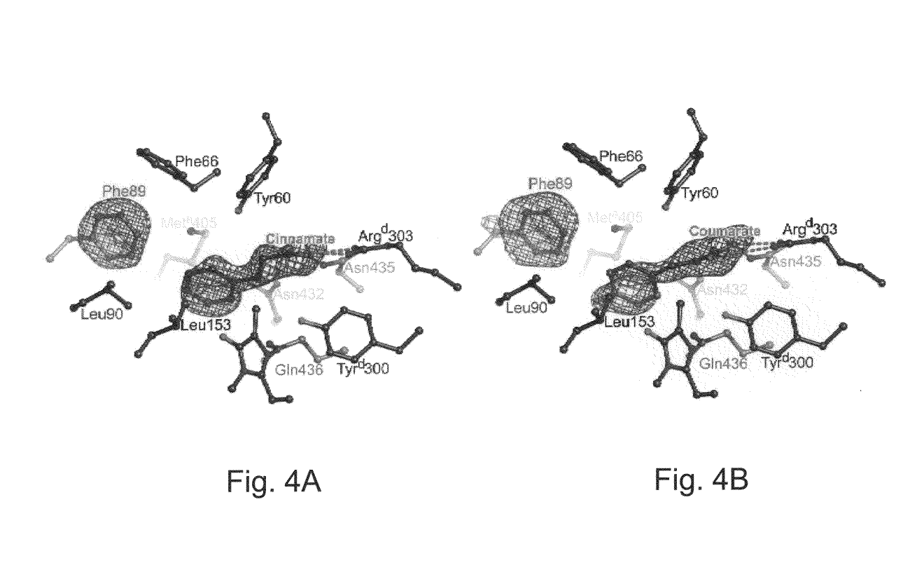

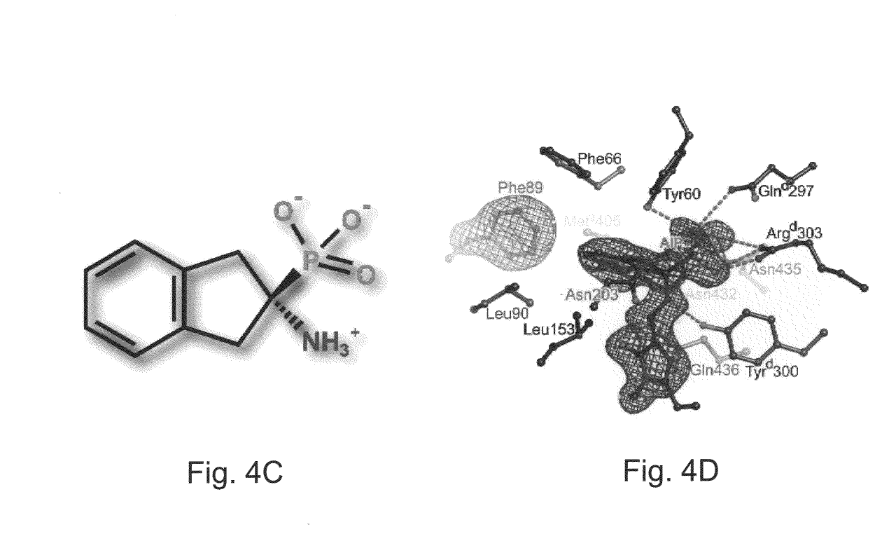

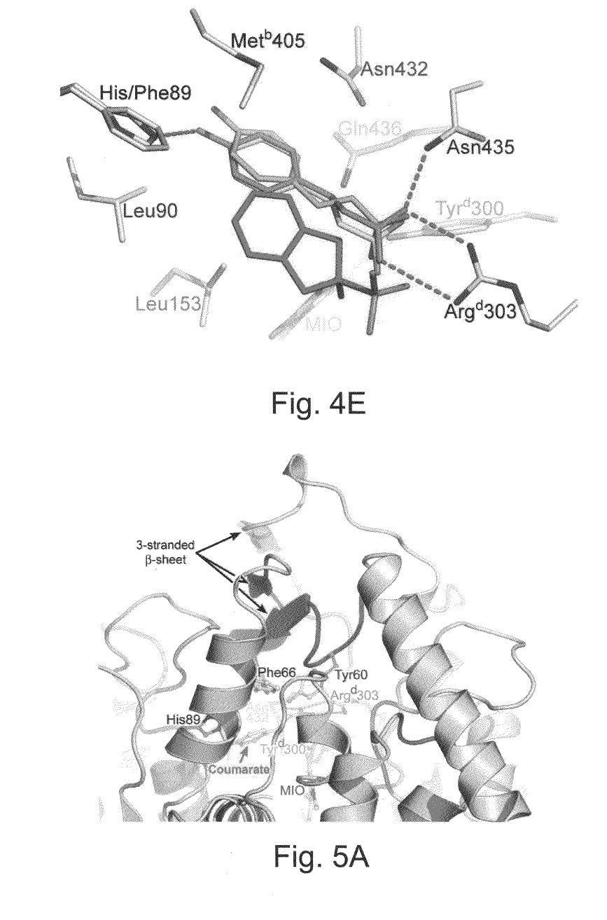

We next determined crystal structures of the H89F mutant complexed with the reaction product cinnamate (FIG. 4A), as well as with coumarate (FIG. 4B) and AIP (FIGS. 4C and 4D). As expected, the phenyl ring of Phe 89 occupies essentially the same space as the His 89 imidazole ring of wild type RsTAL with little active site perturbation. Likewise, cinnamate is coincident with the analogous portion of the coumarate molecule bound to wild type RsTAL. The phenyl rings of cinnamate and Phe 89 are roughly coplanar and within van der Waals distances. Unexpectedly, binding of coumarate to the H89F mutant was observed in crystals. The position and orientation of the bound coumarate differs from that observed in wild type RsTAL relieving expected steric clashes with protein side-chains (FIG. 4E). Moreover, electron-density maps indicate that the coumarate molecule is poorly ordered in the H89F RsTAL active site. With the H89F mutant, although the salt-bridge interaction between the coumarate carboxylate group and the 6-guanido moiety of Arg d303 is preserved, the p-hydroxyl group is pushed away from the phenyl ring of Phe 89 and lacks a hydrogen-bonding partner. Moreover, the opposite face of the propenoate portion of coumarate is oriented toward the MIO co-factor (FIG. 4B). This disrupted binding mode likely explains the inactivity of the H89F RsTAL mutant with L-Tyr as substrate.

In the complex of the H89F mutant with AIP, the amino group of the inhibitor covalently attaches to the methylidene carbon of the MIO co-factor (FIGS. 4C and 4D). Analogous to the cinnamate complex (FIG. 4A), the salt bridge between the Arg^{d}303 δ-guanido moiety and the carboxylate mimic, namely the phosphonate group of ALP, as well as the edge-edge interaction between the phenyl rings of AIP and Phe 89 occur in a conserved fashion. However, because of the covalent attachment of the AIP moiety to the co-factor, the relative positions of AIP atoms with respect to TAL residues differ for AIP in comparison to cinnamate (FIGS. 4A and 4D). Furthermore, the formation of the covalent AIP adduct causes a significant rearrangement of a nearby segment of the polypeptide-chain spanning residues 194 to 205, and points to important mechanistic roles for some of these residues.

TABLE 2
Kinetic parameters for wild type and H89F R. sphaeroides
TAL and comparison with other TAL/PAL enzymes

| | Sub- | | | k_{cat}/Kₘ | AIP |
|---|---|---|---|---|---|
| Enzyme | strate | Kₘ (μM) | k_{cat} (s⁻¹) | (s⁻¹ μM⁻¹) | Kᵢ (μM) |
| wt RsTAL | L-Tyr | 74.2 | 4.32 | 0.058 | NM |
| | L-Phe | 11 400 | 13.10 | 0.0011 | 16.3 |
| H89F RsTAL | L-Tyr | NM | | | NM |
| | L-Phe | 434 | 8.15 | 0.019 | 0.60 |
| RcTAL | L-Tyr | 15.6 | 27.7 | 1.77 | ND |
| | L-Phe | 1277 | 15.1 | 0.0118 | ND |
| SeTAL | L-Tyr | 15.5 | 0.015 | 9.68 × 10⁻⁴ | ND |
| | L-Phe | 2 860 | 0.0038 | 1.3 × 10⁻⁶ | ND |
| ZmPAL | L-Tyr | 19 | 0.9 | 0.0473 | ND |
| | L-Phe | 270 | 10 | 0.037 | ND |
| PcPAL | L-Tyr | 2500 | 0.3 | 1.2 × 10⁻⁴ | ND |
| | L-Phe | 17.2 | 22 | 1.28 | 0.025 |
| SmPAL | L-Phe | 23 | 0.0048 | 2.1 × 10⁻³ | 1.81 |
| PlPAL | L-Phe | 320 | 0.8 | 2.5 × 10⁻³ | ND |

Rc, Se, Zm, Pc, Sm and Pl are R. capsulatus, S. espanaensis, Z. mays, P. crispum, S. maritimus and P. luminescens, respectively. NM is not measurable and ND is not determined.

Active-Site Loops of R. sphaeroides TAL

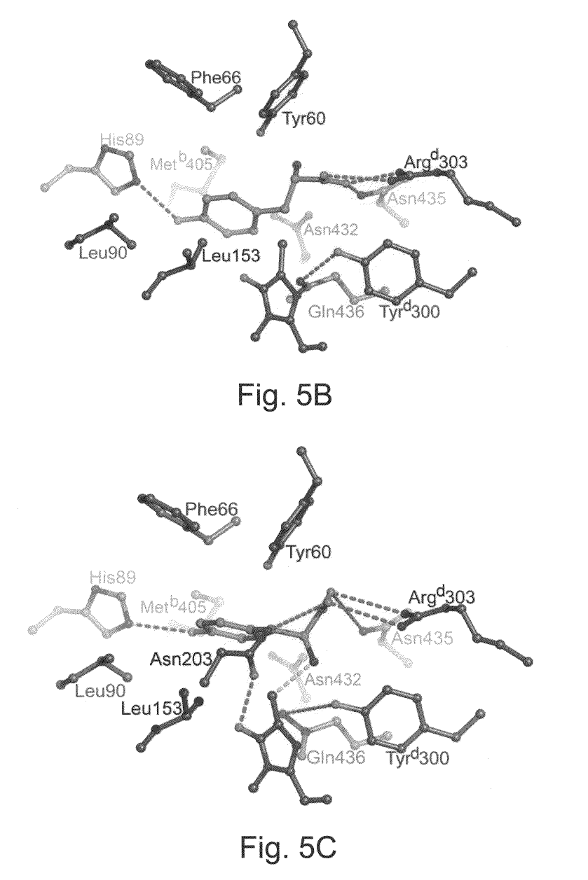

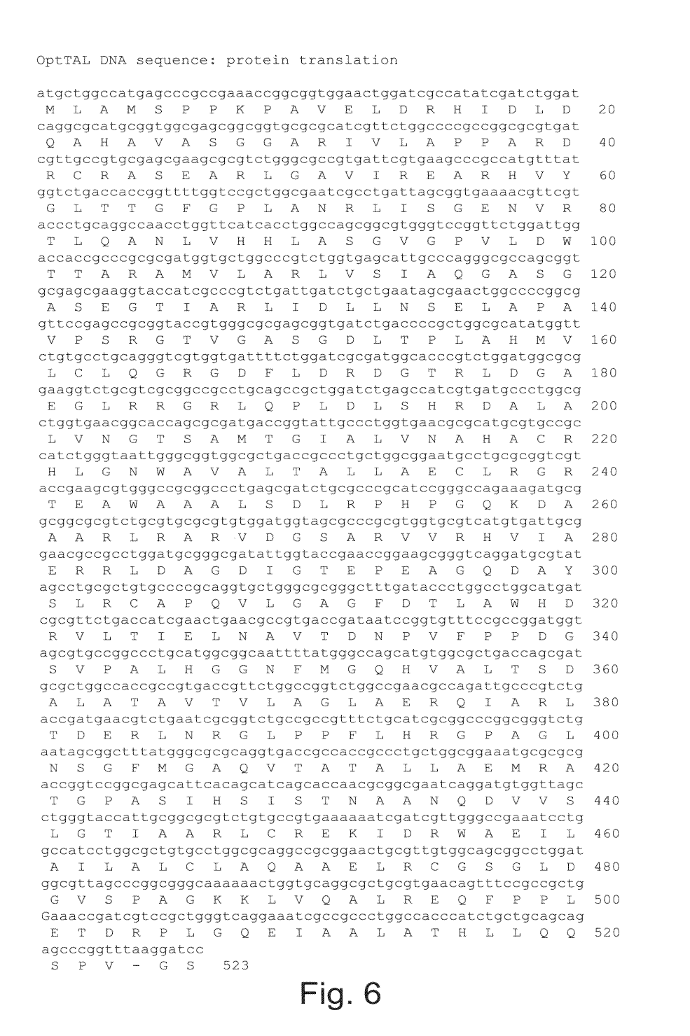

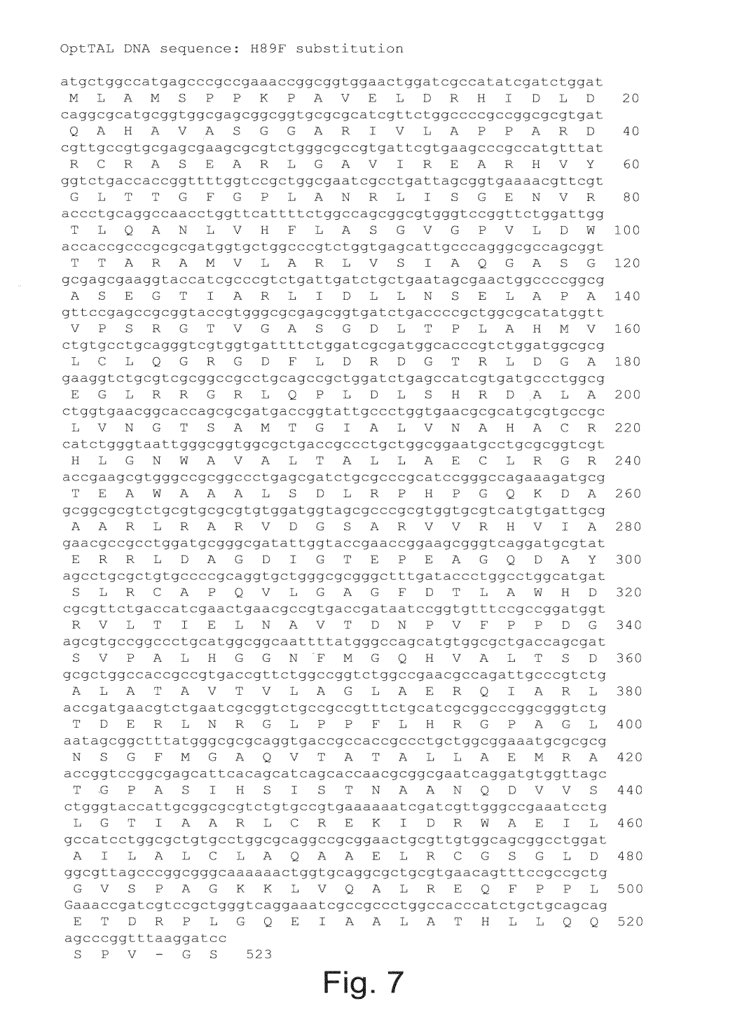

In previously published HAL and PAL crystal structures, two loops situated near the entrance to the active-sites exhibit high mobility, as evidenced by the comparative variability in observed loop conformations, high crystallographic temperature factors, complete disorder and the presence of proteolytically sensitive cleavage sites [13, 17-19]. Flexibility of these loops has been suggested to be a functional requirement for substrate binding and catalysis [20]. One active-site loop, termed the inner lid-loop, originates from the MIO domain of the same polypeptide chain that provides the MIO co-factor, and contains a number of highly conserved residues. The second loop (the outer lid-loop) projects from the C-terminal domain of a dyad-related monomer in the homotetramer. The RsTAL structure is unique in that these lid loops are not only well ordered, but form a compact arrangement within the active-site cavity (FIGS. 2C and 5A).

Three Gly residues, 61, 65, and 67, facilitate the formation of several tight interactions in this region, including a short three-stranded β-sheet, which involves polypeptide segments from both the inner and outer lid-loops (FIG. 5A). The environment of the MIO co-factor in RsTAL is therefore sequestered from the bulk solvent, in contrast to the relatively open active-site access observed in other aromatic amino acid ammonia lyase structures with disordered or more externally positioned lid loops. In the structure of the RsTAL-coumarate complex, the inner lid-loop comes in close contact with the coumarate molecule (FIGS. 2C and 3B).

In previous crystallographic studies of aromatic amino acid ammonia lyases, attempts to obtain complexes with substrate or product were largely unsuccessful. Our observation of well-ordered coumarate binding in RsTAL crystals is likely due to the functionally competent positioning of the lid loops. Without limitation to any particular mechanism, substrate binding may also contribute to stabilizing this arrangement. Thus, the internalized position of the inner-lid loop within an enzyme-substrate complex would explain the observation that L-Tyr inhibits proteolytic cleavage at two sites along this loop in R. toruloides PAL [17]. A comparison of the crystal structures of unliganded and coumarate-complexed RsTAL indicates that coumarate binding is accommodated within the active site with no significant structural perturbation to the surrounding protein. This observation is somewhat surprising, in light of the close packing of residues around the coumarate product and the closed lid-loops which apparently occlude access to the active-site pocket. Without limitation to any particular mechanism, these results suggest a dynamic role of the active-site lid loops in substrate binding, sequestering of reaction intermediates, and/or catalysis.

Implications for the Reaction Mechanism of R. sphaeroides TAL

The structures of the RsTAL-coumarate, -caffeate and -cinnamate complexes support the straightforward structural modeling of the binding of the substrate L-Tyr (FIGS. 5B and 5C). The three-dimensional model of TAL complexed with the substrate L-Tyr forms a new framework for probing the mechanism of TAL and the related aromatic amino acid ammonia lyases and aminomutases.

The first question that can be addressed is which of two proposed reaction mechanisms is more consistent with the modeled substrate-binding mode. Because both the L-Tyr substrate's α-amino group and hydroxyphenyl ring are too distant from the co-factor's electrophilic methylidene-carbon to form a covalent bond, it is evident that the relative positioning of the enzyme and substrate required to initiate the catalytic reaction likely differ to some degree from the arrangement modeled rigidly on the basis of the reaction product complexes. Accommodating substrate movement is indicated by both the availability of space in the vicinity of the substrate binding-site and the much closer approach of the AIP inhibitor to the MIO co-factor (FIGS. 3D and 4D). Nevertheless, it appears that the sizable translational shift of the substrate required to bring the L-Tyr ring Cδ atom (C2 relative to C4 bearing the phenolic hydroxyl moiety) within bonding distance of the MIO methylidene, as suggested by the Friedel-Crafts type mechanism [1], would necessarily disrupt the interactions formed by the α-carboxylate and para-hydroxyl groups to TAL (FIG. 3B). On the other hand, although the substrate's α-amino group in the modeled complex is oriented away from the co-factor, a modest change in conformation of the propenoate group would position the α-amino group appropriately for attack on the MIO methylidene. Most significantly, a re-organization such as this seems quite possible and would not disrupt the anchoring substrate-TAL interactions to each end of the L-Tyr substrate (FIGS. 5B and 5C).

Therefore, without intending to be limited to any particular mechanism, modeling of the enzyme-substrate complex, together with the observed covalent addition of the AIP amino-group to the MIO cofactor and the resultant specificity of the interactions with the TAL, more directly support a mechanism that begins with attack on the MIO methylidene-carbon by the substrate's α-amino group (FIG. 5C). The negative charge formed on the MIO carbonyl oxygen (O2) is stabilized by water-mediated protonation (enol tautomer) in the oxyanion hole (FIG. 2D) and a newly formed interaction with the side-chain amide of Asn 203 (FIG. 5C). Notably, Asn 203 is brought into proximity of the cofactor by a rearrangement of the segment of polypeptide-chain spanning residues 194 to 205 (as observed in the TAL-AIP covalent complex) (FIG. 4D). Substitution of this highly conserved residue by Ala causes large decreases in k_{cat} in both HAL and PAL [24, 25]. The hydroxyl group of Tyr 60 is suitably positioned to abstract the substrate's pro-S β-proton (brown in FIG. 1), which is oriented anti-periplanar to the α-amino group. Elimination of the α-amino group then yields the first product, an aryl-acid bearing a trans α,β-double bond within the propenoate moiety, and an ammonia adduct with MIO. Due to steric interactions with the active site including the MIO-ammonia adduct, the propenoate moiety would undergo a conformational adjustment as observed in the coumarate (FIG. 3B) and cinnamate (FIG. 4A) complexes described above. This product retention (aryl-acid and MIO-ammonia adduct) hypothesis may also explain the ability of the mechanistically and structurally related aminomutases to catalyze amino group recapture at the propenoate carbon of the aryl-acid product equivalent to the β-position on α-amino acid substrates to form β-amino acids (FIG. 1).

In the observed conformation of the bound coumarate product, the non-co-planarity between the propenoate carbon-carbon double bond and the carboxylate group would disfavor Michael addition of ammonia to the 3-carbon (the aminomutase reaction shown in FIG. 1). Without limitation to any particular mechanism, modulation of the relative orientations of the alkene and carboxylate groups may serve as a key determinant of the reaction course (and consequently product specificity) of the MIO-dependent ammonia lyases and aminomutases.

Further Details

The crystal structure of RsTAL complexed with coumarate provides the first definitive characterization of substrate or product binding to any aromatic amino-acid ammonia-lyase. The binding of the coumarate molecule within the active site of RsTAL involves interactions of the propenoate moiety with protein residues that are highly conserved among the aromatic amino-acid ammonia-lyases, for example Tyr 60 (putative catalytic base) and Arg 303 (carboxylate tail recognition). On the other hand, the residues that interact with the aromatic ring are more variable, as expected given the differences in side-chain selectivities within the larger family of MIO-dependent enzymes. Most notably, the RsTAL His89-imidazole group, which hydrogen bonds with the coumarate p-hydroxyl moiety, plays a critical role in discriminating between L-Tyr and L-Phe or L-His as substrates. This discovery is the long sought selectivity filter in PAL/TAL/HALs. Replacement of His 89 by Phe, a residue more characteristic of the PALs, yields a mutant RsTAL with a marked switch in substrate preference from L-Tyr to L-Phe. Structures of the H89F mutant RsTAL in complex with the reaction product, cinnamic acid, or the PAL inhibitor, 2-aminoindan-2-phosphonic acid, revealed binding modes in which the phenyl rings of Phe 89 and the ligands interact edge to edge. Based upon a comparison of available X-ray crystal structures, it appears that two loops capping the active site of the aromatic amino-acid ammonia-lyases can play a dynamic role in both substrate binding and catalysis. Our combined structural and functional studies provide a near atomic resolution basis for understanding the reaction mechanism of this enzyme family and also the aromatic amino-acid aminomutases, which biosynthesize β-amino acids in nature. By identifying key residues and regions of the enzymes, our structural and functional studies facilitate engineering of specificity determinants in aromatic amino-acid ammonia-lyases and the related aminomutases, using both site-directed approaches and focused combinatorial methods to expand the technological utility of these MIO-dependent enzymes.

### Additional Details Regarding Experimental Procedures

Synthetic Gene, Expression, Purification and Mutagenesis of R. sphaeroides TAL

A synthetic gene encoding the amino acid sequence of R. sphaeroides TAL (RsTAL) was synthesized by GenScript (www (dot) genscript (dot) com). The gene sequence was optimized for codon preferences in Escherichia coli and bracketed by 5′-NcoI and 3′-BamHI restriction sites. The gene was inserted between the NcoI and BamHI sites of the expression vector pHIS8, which, under the control of a T7 promoter, yields the target protein fused to a thrombin-cleavable N-terminal octahistidine tag [26]. For heterologous expression of RsTAL, the plasmid pHIS8-RsTAL was transformed into the expression host E. coli BL21(DE3) (Novagen), expression was induced with isopropyl-β-D-thiogalactoside, and RsTAL was purified by nickel-nitrilotriacetic-acid (NTA) coupled agarose chromatography followed by gel filtration chromatography after thrombin cleavage and removal of the His8 tag.

Briefly, E. coli cultures in TB medium were grown at 37° C. to an optical density (600 nm) of 1.5, induced with 1 mM isopropyl-β-D-thiogalactoside, and allowed to grow for an additional 6 hrs at 20° C. Bacterial cells were harvested by centrifugation, resuspended in lysis buffer (50 mM Tris HCl, pH 8.0, 0.5 M NaCl, 20 mM imidazole, 1% (v/v) Tween20, 10% (v/v) glycerol and 20 mM 2-mercaptoethanol) and lysed by sonication. RsTAL was isolated from the E. coli lysate by affinity chromatography with Ni²⁺-NTA agarose and eluted with lysis buffer supplemented with 0.25 M imidazole. Nearly homogeneous RsTAL was treated with thrombin for cleavage of the octahistidine tag, and then further purified by gel-exclusion chromatography using a Superdex 200 HR26/60 column (Pharmacia Biosystems). Site-directed mutants of the RsTAL gene were created in the plasmid pHIS8-RsTAL using the QuikChange protocol (Stratagene), and mutant proteins were expressed and purified as described for wild type RsTAL. The DNA sequence of the mutant construct was confirmed by sequencing of the entire RsTAL gene insert in both the forward and reverse directions.

Enzyme Assays

TAL activity was measured spectrophotometrically by monitoring the formation of a conjugated aryl-acid product. The conversion of L-Tyr to p-coumarate was followed at 310 nm and L-Phe to cinnamate at 280 nm. The assay mixture (total volume 0.5 ml) contained 0.1 M Tris HCl (pH 8.5), and for each fixed amount of TAL, eight different initial concentrations of amino acid substrate. After pre-incubation of the enzyme at 37° C. for 2 min, reactions were initiated by the addition of substrate, and formation of product was monitored for 5 min. For wild type RsTAL, activity was assayed with 7.5 μg protein and initial L-Tyr concentrations between 0.01 and 2.4 mM, and PAL activity was assayed with 30 μg protein and initial L-Phe concentrations between 3.2 and 51.2 mM. For the RsTAL H89F mutant, PAL activity was assayed with 10 μg protein and initial L-Phe concentrations between 0.05 and 5.2 mM. Each series of assays were performed in triplicate, and a Hanes plot was used for the estimation of steady-state kinetic constants.

The inhibition of enzyme activity by AIP was measured with a similar assay system, except that the enzyme was pre-incubated for 7 min in the presence of fixed concentrations of AIP. Inhibition of PAL activity of wild type RsTAL was measured with 15 μg protein, six AIP concentrations between 0 and 20 μM, and initial L-Phe concentrations between 9.6 and 25.6 mM. Inhibition of PAL activity of the H89F mutant was measured with 10 μg protein, five AIP concentrations between 0 and 5 μM, with initial L-Phe concentrations between 0.4 and 1.6 mM. Duplicate sets of enzyme assays were performed and a Dixon plot was used for the estimation of the AIP inhibition constant, Kᵢ. AIP was a gift from Dr. Jerzy Zon, Wroclaw University, Poland.

Crystallization and X-Ray Structure Elucidation of R. sphaeroides TAL

Monoclinic crystals of RsTAL (space group P2%) were grown from a 1:1 mixture of protein solution (20 mg/ml in 12.5 mM Tris-HCl, pH 7.5, 50 mM NaCl) and a reservoir solution (0.1 M MOPSO—Na⁺, pH 7.0, 7% (w/v) polyethylene glycol 8000, 0.3 M ammonium acetate, 2 mM dithiothreitol, 35 mM cyclohexylbutanoyl-N-hydroxyethylglucamide) using vapor diffusion against reservoir solutions at 4° C. Crystal growth typically occurred over a period of one to three weeks and was expedited through microseeding. The monoclinic crystals exhibit a rhomboid morphology and grow to a maximum size of 0.4×0.1×0.1 mm. Crystals of RsTAL in complex with small molecule ligands were produced by soaking crystals for 24-48 hrs in reservoir solutions supplemented with 10-20 mM coumaric acid, caffeic acid, cinnamic acid or AIP.

Crystals were transferred briefly to a cryo-protectant solution (consisting of reservoir solution supplemented with 15-20% (v/v) polyethylene glycol 400) prior to immersion in liquid nitrogen. X-ray diffraction data were measured from frozen crystals at beam lines 8.2.1 and 8.2.2 of the Advanced Light Source (Lawrence Berkeley National Laboratory) on an ADSC Quantum 210 CCD detector or at beam line 1-5 of the Stanford Synchrotron Radiation Laboratory on an ADSC Quantum 315 CCD detector. Diffraction intensities were indexed, integrated, and scaled with the programs XDS and XSCALE [27], or Mosflm [28] and Scala [29] and are summarized in Table 1.

RsTAL crystals contain two complete homotetramers per asymmetric unit and diffract up to 1.58 Å resolution. Initial phases were determined by molecular-replacement using the program Molrep [30]. A homology model for RsTAL was constructed with the program Modeller [31], based on the structure of P. putida HAL (PDB entry 1 GKM). The program ARP/wARP [32] was used for automated rebuilding of the initial structure using an eight-fold, non-crystallographic symmetry (NCS) averaged map. Subsequent structural refinement used the program CNS [33] with NCS restraints applied until the final stages. Xfit [34] was used for map inspection and manual rebuilding of the atomic model. Programs from the CCP4 suite [29] were employed for all other crystallographic calculations. For refinement of the coumarate, caffeate and cinnamate molecules, no conformational restraints were applied to the freely rotatable dihedral angles of the propenoate moiety or to enforce similarity of NCS-related copies. Figures were drawn with the program Pymol (Delano Scientific, San Carlos, Calif.).

Relevant crystal structure information has been deposited. The atomic coordinates and structure factors have been deposited in the Protein Data Bank, www.pdb.org. PDB ID code 2o6y provides the atomic coordinates for RsTAL (Tyrosine ammonia-lyase from Rhodobacter sphaeroides). PDB ID code 2o7b provides the atomic coordinates and structure factors for RsTAL-coumarate (Tyrosine ammonia-lyase from Rhodobacter sphaeroides, complexed with coumarate). PDB ID code 2o7d provides the atomic coordinates and structure factors for RsTAL-caffeate (Tyrosine ammonia-lyase from Rhodobacter sphaeroides, complexed with caffeate). PDB ID code 2o78 provides the atomic coordinates and structure factors for H89F RsTAL-cinnamate (Tyrosine ammonia-lyase from Rhodobacter sphaeroides (His89Phe variant) complexed with cinnamic acid). PDB ID code 2o7f provides the atomic coordinates and structure factors for H89F RsTAL-coumarate (Tyrosine ammonia-lyase from Rhodobacter sphaeroides (His89Phe variant), complexed with coumaric acid). PDB ID code 2o7e provides the atomic coordinates and structure factors for H89F RsTAL-AIP (Tyrosine ammonia-lyase from Rhodobacter sphaeroides (His89Phe variant), bound to 2-aminoindan-2-phosphonic acid). For further details, see also, Louie et al. “Structural Determinants and Modulation of Substrate Specificity in Phenyalanine-Tyrosine Ammonia-Lyases” Chemistry and Biology 13, 1327-1338 (2006), incorporated herein by reference in its entirety. Atomic coordinates and structure factors were also provided on CD-ROM in U.S. Ser. No. 60/872,162 SUBSTRATE SWITCHED AMMONIA LYSASES AND MUTASES by Noel et al., filed Dec. 1, 2006; U.S. Ser. No. 60/873,668 SUBSTRATE SWITCHED AMMONIA LYSASES AND MUTASES by Noel et al., filed Dec. 6, 2006; and U.S. Ser. No. 60/874,709 SUBSTRATE SWITCHED AMMONIA LYSASES AND MUTASES by Noel et al., filed Dec. 12, 2006, all incorporated herein by reference in their entirety for all purposes.

### REFERENCES

1. Poppe, L. & Rétey, J. (2005). Friedel-Crafts-type mechanism for the enzymatic elimination of ammonia from histidine and phenylalanine. Angew Chem. Int. Ed. Engl. 44, 3668-3688.
2. Rosler, J., Krekel, F., Amrhein, N. & Schmid, J. (1997). Maize phenylalanine ammonia-lyase has tyrosine ammonia-lyase activity. Plant Physiol. 113, 175-179.
3. Jiang, H., Wood, K. V. & Morgan, J. A. (2005). Metabolic engineering of the phenylpropanoid pathway in Saccharomyces cerevisiae. Appl. Environ. Microbiol. 71, 2962-2969.
4. Xiang, L. & Moore, B. S. (2005). Biochemical characterization of a prokaryotic phenylalanine ammonia lyase. J. Bacteriol. 187, 4286-4289.
5. Williams, J. S., Thomas, M. & Clarke, D. J. (2005). The gene stlA encodes a phenylalanine ammonia-lyase that is involved in the production of a stilbene antibiotic in Photorhabdus luminescens TT01. Microbiology 151, 2543-2550.
6. Hill, A. M., Thompson, B. L., Harris, J. P. & Segret, R. (2003). Investigation of the early stages in soraphen A biosynthesis. Chem. Biochem. 4, 1358-1359.
7. Emes, A. V. & Vining, L. C. (1970). Partial purification and properties of 1-phenylalanine ammonia lyase from Streptomyces verticillatus. Can. J. Biochem. 48, 613-622.
8. Kyndt, J. A., Meyer, T. E., Cusanovich, M. A. & Van Beeumen, J. J. (2002). Characterization of a bacterial tyrosine ammonia lyase, a biosynthetic enzyme for the photoactive yellow protein. FEBS Lett. 512, 240-244.
9. Watts, K. T., Lee, P. C. & Schmidt-Dannert, C. (2004). Exploring recombinant flavonoid biosynthesis in metabolically engineered Escherichia coli. Chem. Biochem. 5, 500-507.
10. Berner, M., Krug, D., Bihlmaier, C., Vente, A., Müller, R., and Bechthold, A. (2006). Genes and enzymes involved in caffeic acid biosynthesis in the actinomycete Saccharothrix espanaensis. J. Bacteriol. 188, 2666-2673.
11. Baedeker, M. & Schulz, G. E. (2002). Autocatalytic peptide cyclization during chain folding of histidine ammonia-lyase. Structure 10, 61-67.
12. Rétey, J. (2003). Discovery and role of methylidene imidazolone, a highly electrophilic prosthetic group. Biochim. Biophys. Acta 1647, 179-184.
13. Calabrese, J. C., Jordan, D. B., Boodhoo, A., Sariaslani, S. & Vannelli, T. (2004). Crystal structure of phenylalanine ammonia lyase: multiple helix dipoles implicated in catalysis. Biochemistry 43, 11403-11416.
14. Christenson, S. D., Liu, W., Toney, M. D. & Shen, B. (2003). A novel 4-methylideneimidazole-5-one-containing tyrosine aminomutase in enediyne antitumor antibiotic C-1027 biosynthesis. J. Am. Chem. Soc. 125, 6062-6063.
15. Walker, K. D., Klettke, K., Akiyama, T. & Croteau, R. (2004). Cloning, heterologous expression, and characterization of a phenylalanine aminomutase involved in taxol biosynthesis. J. Biol. Chem. 279, 53947-53954.
16. Steele, C. L., Chen, Y., Dougherty, B. A., Li, W., Hofstead, S., Lam, K. S., Xing, Z. & Chiang, S. J. (2005). Purification, cloning, and functional expression of phenylalanine aminomutase: the first committed step in Taxol side-chain biosynthesis. Arch. Biochem. Biophys. 438, 1-10.
17. Wang, L., Gamez, A., Sarkissian, C. N., Straub, M., Patch, M. G., Han, G. W., Striepeke, S., Fitzpatrick, P., Scriver, C. R. & Stevens, R. C. (2005). Structure-based chemical modification strategy for enzyme replacement treatment of phenylketonuria. Mol. Genet. Metab. 86, 134-140.
18. Ritter, H. & Schulz, G. E. (2004). Structural basis for the entrance into the phenylpropanoid metabolism catalyzed by phenylalanine ammonia-lyase. Plant Cell 16, 3426-3436.
19. Schwede, T. F., Rétey, J. & Schulz, G. E. (1999). Crystal structure of histidine ammonia-lyase revealing a novel polypeptide modification as the catalytic electrophile. Biochemistry 38, 5355-5361.
20. Pilbák, S., Tomin, A., Rétey, J., and Poppe, L. (2006). The essential tyrosine-containing loop conformation and the role of the C-terminal multi-helix region in eukaryotic phenylalanine ammonia-lyases. FEBS J. 273, 1004-1019.
21. Baedeker, M. & Schulz, G. E. (2002). Structures of two histidine ammonia-lyase modifications and implications for the catalytic mechanism. Eur. J. Biochem. 269, 1790-1797.
22. Appert, C., Logemann, E., Hahlbrock, K., Schmid, J. & Amrhein, N. (1994). Structural and catalytic properties of the four phenylalanine ammonia-lyases from parsley (Petroselinum crispum Nym). Eur. J. Biochem. 225, 2177-2185.
23. Appert, C., Zón, J. & Amrhein, N. (2003). Kinetic analysis of the inhibition of phenylalanine ammonia-lyase by 2-aminoindan-2-phosphonic acid and other phenylalanine analogues. Phytochem. 62, 415-422.
24. Rother, D., Poppe, L., Viergutz, S., Langer, B. & Rétey, J. (2001). Characterization of the active site of histidine ammonia-lyase from Pseudomonas putida. Eur. J. Biochem. 268, 6011-6019.
25. Rother, D., Poppe, L., Morlock, G., Viergutz, S. & Rétey, J. (2002). An active site homology model of phenylalanine ammonia-lyase from Petroselinum crispum. Eur. J. Biochem. 269, 3065-3075.
26. Jez, J. M., Ferrer, J. L., Bowman, M. E., Dixon, R. A. & Noel, J. P. (2000). Dissection of malonyl-coenzyme A decarboxylation from polyketide formation in the reaction mechanism of a plant polyketide synthase. Biochemistry 39, 890-902.
27. Kabsch, W. (1993). Automatic processing of rotation diffraction data from crystals of initially unknown symmetry and cell constants. J. Appl. Crystallog. 26, 795-800.
28. Leslie, A. G. W. (1992). Joint CCP4+ESF-EAMCB Newsletter on Protein Crystallography, No. 26.
29. Collaborative Computational Project Number 4. (1994). The CCP4 suite: programs for protein crystallography. Acta Crystallog. D50, 760-763.
30. Vagin, A. & Teplyakov, A. (1997). MOLREP: an automated program for molecular replacement. J. Appl. Crystallog. 30, 1022-1025.
31. Sali, A. & Blundell, T. L. (1993). Comparative protein modelling by satisfaction of spatial restraints. J. Mol. Biol. 234, 779-815.
32. Lamzin, V. S. & Wilson, K. S. (1993). Automated refinement of protein models. Acta Crystallog. D49, 129-149.
33. Brunger, A. T. & Warren, G. L. (1998). Crystallography and NMR system: a new software suite for macromolecular structure determination. Acta Crystallog. D54, 905-921.
34. McRee, D. E. (1999). XtalView/Xfit: a versatile program for manipulating atomic coordinates and electron density. J. Struct. Biol. 125, 156-165.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A recombinant amino acid ammonia lyase enzyme, comprising at least one mutation in an active site of the enzyme, wherein the mutation switches substrate preference of the lyase enzyme from a first substrate to a second substrate.

2. The recombinant amino acid ammonia lyase enzyme of claim 1, wherein the first substrate is an amino acid, and the second substrate is an amino acid.

3. The recombinant amino acid ammonia lyase enzyme of claim 2, wherein the first and second amino acids are aromatic amino acids.

4. The recombinant amino acid ammonia lyase enzyme of claim 2, wherein the first and second amino acids are unnatural or rare amino acids.

5. The recombinant amino acid ammonia lyase enzyme of claim 3, wherein the first amino acid is tyrosine or histidine and the second amino acid is phenylalanine.

6. The recombinant amino acid ammonia lyase enzyme of claim 1, wherein the recombinant enzyme is derived from a tyrosine or histidine ammonia lyase, and wherein the recombinant enzyme preferentially deaminates L-Phe.

7. The recombinant amino acid ammonia lyase enzyme of claim 1, wherein the mutation is in a residue corresponding to His 89 of Rhodobacter sphaeroides Tyrosine Ammonia Lyase.

8. The recombinant amino acid ammonia lyase enzyme of claim 1, wherein the enzyme comprises a 4-methylidene-imidazole-5-one (MOI) cofactor prosthetic group.

9. The recombinant amino acid ammonia lyase enzyme of claim 1, wherein the enzyme produces trans-cinnamic acid.

10. A nucleic acid that encodes the recombinant amino acid ammonia lyase enzyme of claim 1.

11. A recombinant cell that comprises the recombinant amino acid ammonia lyase enzyme of claim 1.

12. The recombinant cell of claim 11, wherein the cell encodes a recombinant tyrosine amino acid-type ammonia lyase enzyme that comprises a mutation converting a kinetic preference of the enzyme for tyrosine into a preference for phenylalanine.

13. The recombinant cell of claim 11, wherein the cell encodes a recombinant tyrosine amino acid-type ammonia lyase enzyme that comprises a mutation converting a kinetic preference of the enzyme for phenylalanine into a preference for tyrosine.

14. The cell of claim 11, wherein the cell a bacterial cell, a fungal cell, a plant cell or an animal cell.

15. The cell of claim 11, wherein the cell displays increased production of trans-cinnamic acid, or of a phenylpropanoid, or both.

16. The cell of claim 15, wherein the phenylpropanoid is selected from the group consisting of: lignins, flavonoids, stilbenes, and coumarins.

17. A library of amino acid ammonia lyase polypeptides, the library comprising:
a plurality of polypeptides comprising or derived from amino acid ammonia lyase enzyme polypeptides, wherein the plurality of polypeptides collectively comprise a plurality of mutations of at least one amino acid in at least one region of the polypeptides, the region corresponding to an active site of an amino acid ammonia lyase enzyme.

18-23. (canceled)

24. A method of modifying a selected enzyme, the method comprising:
accessing an information set derived from a crystal structure of an amino acid lyase enzyme, or of a homologue thereof, complexed with a product, and, based on information in the information set, predicting whether making a change to the structure of the enzyme will alter an interaction between a substrate, intermediate or product and the enzyme; and,
modifying the enzyme based upon on said predicting.

25-29. (canceled)

30. A method of modifying a selected enzyme, the method comprising:
accessing an information set derived from a crystal structure of a tyrosine ammonia lyase enzyme, or a homologue thereof, and, based on information in the information set, predicting whether making a change to the structure of the enzyme will alter an interaction between a substrate of the enzyme, or of a product produced by the enzyme; and,
modifying the enzyme based upon on said predicting.

31-36. (canceled)

37. A method of deaminating L-DOPA, comprising contacting L-DOPA with a purified or recombinant tyrosine ammonia lyase enzyme.


SEQUENCE LISTING (40 sequences)

SEQ ID NO: 1 - DNA, 1578 bp, Rhodobacter sphaeroides
atgctggccatgagcccgccgaaaccggcggtggaactggatcgccatatcgatctggatcaggcgcatgcggtggcgagcggcggtgcgcgcatcgttctggccccgccggcgcgtgatcgttgccgtgcgagcgaagcgcgtctgggcgccgtgattcgtgaagcccgccatgtttatggtctgaccaccggttttggtccgctggcgaatcgcctgattagcggtgaaaacgttcgtaccctgcaggccaacctggttcatcacctggccagcggcgtgggtccggttctggattggaccaccgcccgcgcgatggtgctggcccgtctggtgagcattgcccagggcgccagcggtgcgagcgaaggtaccatcgcccgtctgattgatctgctgaatagcgaactggccccggcggttccgagccgcggtaccgtgggcgcgagcggtgatctgaccccgctggcgcatatggttctgtgcctgcagggtcgtggtgattttctggatcgcgatggcacccgtctggatggcgcggaaggtctgcgtcgcggccgcctgcagccgctggatctgagccatcgtgatgccctggcgctggtgaacggcaccagcgcgatgaccggtattgccctggtgaacgcgcatgcgtgccgccatctgggtaattgggcggtggcgctgaccgccctgctggcggaatgcctgcgcggtcgtaccgaagcgtgggccgcggccctgagcgatctgcgcccgcatccgggccagaaagatgcggcggcgcgtctgcgtgcgcgtgtggatggtagcgcccgcgtggtgcgtcatgtgattgcggaacgccgcctggatgcgggcgatattggtaccgaaccggaagcgggtcaggatgcgtatagcctgcgctgtgccccgcaggtgctgggcgcgggctttgataccctggcctggcatgatcgcgttctgaccatcgaactgaacgccgtgaccgataatccggtgtttccgccggatggtagcgtgccggccctgcatggcggcaattttatgggccagcatgtggcgctgaccagcgatgcgctggccaccgccgtgaccgttctggccggtctggccgaacgccagattgcccgtctgaccgatgaacgtctgaatcgcggtctgccgccgtttctgcatcgcggcccggcgggtctgaatagcggctttatgggcgcgcaggtgaccgccaccgccctgctggcggaaatgcgcgcgaccggtccggcgagcattcacagcatcagcaccaacgcggcgaatcaggatgtggttagcctgggtaccattgcggcgcgtctgtgccgtgaaaaaatcgatcgttgggccgaaatcctggccatcctggcgctgtgcctggcgcaggccgcggaactgcgttgtggcagcggcctggatggcgttagcccggcgggcaaaaaactggtgcaggcgctgcgtgaacagtttccgccgctggaaaccgatcgtccgctgggtcaggaaatcgccgccctggccacccatctgctgcagcagagcccggtttaaggatcc

SEQ ID NO: 2 - protein, 523 aa, Rhodobacter sphaeroides
MLAMSPPKPAVELDRHIDLDQAHAVASGGARIVLAPPARDRCRASEARLGAVIREARHVYGLTTGFGPLANRLISGENVRTLQANLVHHLASGVGPVLDWTTARAMVLARLVSIAQGASGASEGTIARLIDLLNSELAPAVPSRGTVGASGDLTPLAHMVLCLQGRGDFLDRDGTRLDGAEGLRRGRLQPLDLSHRDALALVNGTSAMTGIALVNAHACRHLGNWAVALTALLAECLRGRTEAWAAALSDLRPHPGQKDAAARLRARVDGSARVVRHVIAERRLDAGDIGTEPEAGQDAYSLRCAPQVLGAGFDTLAWHDRVLTIELNAVTDNPVFPPDGSVPALHGGNFMGQHVALTSDALATAVTVLAGLAERQIARLTDERLNRGLPPFLHRGPAGLNSGFMGAQVTATALLAEMRATGPASIHSISTNAANQDVVSLGTIAARLCREKIDRWAEILAILALCLAQAAELRCGSGLDGVSPAGKKLVQALREQFPPLETDRPLGQEIAALATHLLQQSPV

SEQ ID NO: 3 - DNA, 1578 bp, Artificial
  H89F mutant of Rhodobacter sphaeroides tyrosine ammonia lyase
atgctggccatgagcccgccgaaaccggcggtggaactggatcgccatatcgatctggatcaggcgcatgcggtggcgagcggcggtgcgcgcatcgttctggccccgccggcgcgtgatcgttgccgtgcgagcgaagcgcgtctgggcgccgtgattcgtgaagcccgccatgtttatggtctgaccaccggttttggtccgctggcgaatcgcctgattagcggtgaaaacgttcgtaccctgcaggccaacctggttcattttctggccagcggcgtgggtccggttctggattggaccaccgcccgcgcgatggtgctggcccgtctggtgagcattgcccagggcgccagcggtgcgagcgaaggtaccatcgcccgtctgattgatctgctgaatagcgaactggccccggcggttccgagccgcggtaccgtgggcgcgagcggtgatctgaccccgctggcgcatatggttctgtgcctgcagggtcgtggtgattttctggatcgcgatggcacccgtctggatggcgcggaaggtctgcgtcgcggccgcctgcagccgctggatctgagccatcgtgatgccctggcgctggtgaacggcaccagcgcgatgaccggtattgccctggtgaacgcgcatgcgtgccgccatctgggtaattgggcggtggcgctgaccgccctgctggcggaatgcctgcgcggtcgtaccgaagcgtgggccgcggccctgagcgatctgcgcccgcatccgggccagaaagatgcggcggcgcgtctgcgtgcgcgtgtggatggtagcgcccgcgtggtgcgtcatgtgattgcggaacgccgcctggatgcgggcgatattggtaccgaaccggaagcgggtcaggatgcgtatagcctgcgctgtgccccgcaggtgctgggcgcgggctttgataccctggcctggcatgatcgcgttctgaccatcgaactgaacgccgtgaccgataatccggtgtttccgccggatggtagcgtgccggccctgcatggcggcaattttatgggccagcatgtggcgctgaccagcgatgcgctggccaccgccgtgaccgttctggccggtctggccgaacgccagattgcccgtctgaccgatgaacgtctgaatcgcggtctgccgccgtttctgcatcgcggcccggcgggtctgaatagcggctttatgggcgcgcaggtgaccgccaccgccctgctggcggaaatgcgcgcgaccggtccggcgagcattcacagcatcagcaccaacgcggcgaatcaggatgtggttagcctgggtaccattgcggcgcgtctgtgccgtgaaaaaatcgatcgttgggccgaaatcctggccatcctggcgctgtgcctggcgcaggccgcggaactgcgttgtggcagcggcctggatggcgttagcccggcgggcaaaaaactggtgcaggcgctgcgtgaacagtttccgccgctggaaaccgatcgtccgctgggtcaggaaatcgccgccctggccacccatctgctgcagcagagcccggtttaaggatcc

SEQ ID NO: 4 - protein, 523 aa, Artificial
  H89F mutant of Rhodobacter sphaeroides tyrosine ammonia lyase
MLAMSPPKPAVELDRHIDLDQAHAVASGGARIVLAPPARDRCRASEARLGAVIREARHVYGLTTGFGPLANRLISGENVRTLQANLVHFLASGVGPVLDWTTARAMVLARLVSIAQGASGASEGTIARLIDLLNSELAPAVPSRGTVGASGDLTPLAHMVLCLQGRGDFLDRDGTRLDGAEGLRRGRLQPLDLSHRDALALVNGTSAMTGIALVNAHACRHLGNWAVALTALLAECLRGRTEAWAAALSDLRPHPGQKDAAARLRARVDGSARVVRHVIAERRLDAGDIGTEPEAGQDAYSLRCAPQVLGAGFDTLAWHDRVLTIELNAVTDNPVFPPDGSVPALHGGNFMGQHVALTSDALATAVTVLAGLAERQIARLTDERLNRGLPPFLHRGPAGLNSGFMGAQVTATALLAEMRATGPASIHSISTNAANQDVVSLGTIAARLCREKIDRWAEILAILALCLAQAAELRCGSGLDGVSPAGKKLVQALREQFPPLETDRPLGQEIAALATHLLQQSPV

SEQ ID NO: 5 - protein, 8 aa, Rhodobacter sphaeroides
YGLTTGFG

SEQ ID NO: 6 - protein, 5 aa, Rhodobacter sphaeroides
LVHHL

SEQ ID NO: 7 - protein, 9 aa, Rhodobacter sphaeroides
TVGASGDLT

SEQ ID NO: 8 - protein, 7 aa, Rhodobacter sphaeroides
QDAYSLR

SEQ ID NO: 9 - protein, 7 aa, Rhodobacter sphaeroides
GGNFMGQ

SEQ ID NO: 10 - protein, 7 aa, Rhodobacter sphaeroides
NAANQDV

SEQ ID NO: 11 - protein, 8 aa, Petroselinum crispum
YGVTTGFG

SEQ ID NO: 12 - protein, 5 aa, Petroselinum crispum
LIRFL

SEQ ID NO: 13 - protein, 9 aa, Petroselinum crispum
TITASGDLV

SEQ ID NO: 14 - protein, 7 aa, Petroselinum crispum
QDRYALR

SEQ ID NO: 15 - protein, 7 aa, Petroselinum crispum
GGNFQGT

SEQ ID NO: 16 - protein, 7 aa, Petroselinum crispum
EQHNQDV

SEQ ID NO: 17 - protein, 8 aa, Zea mays
YGVTTGFG

SEQ ID NO: 18 - protein, 5 aa, Zea mays
LLRHL

SEQ ID NO: 19 - protein, 9 aa, Zea mays
TITASGDLV

SEQ ID NO: 20 - protein, 7 aa, Zea mays
QDRYALR

SEQ ID NO: 21 - protein, 7 aa, Zea mays
GGNFQGT

SEQ ID NO: 22 - protein, 7 aa, Zea mays
DEHNQDV

SEQ ID NO: 23 - protein, 8 aa, Rhodosporidium toruloides
YGVTTGFG

SEQ ID NO: 24 - protein, 5 aa, Rhodosporidium toruloides
LLEHQ

SEQ ID NO: 25 - protein, 9 aa, Rhodosporidium toruloides
TISASGDLS

SEQ ID NO: 26 - protein, 7 aa, Rhodosporidium toruloides
QDRYPLR

SEQ ID NO: 27 - protein, 7 aa, Rhodosporidium toruloides
GGNFQAA

SEQ ID NO: 28 - protein, 7 aa, Rhodosporidium toruloides
EMANQAV

SEQ ID NO: 29 - protein, 8 aa, Anabaena variabilis
YGVTSGFG

SEQ ID NO: 30 - protein, 5 aa, Anabaena variabilis
LVWFL

SEQ ID NO: 31 - protein, 9 aa, Anabaena variabilis
SIGASGDLV

SEQ ID NO: 32 - protein, 7 aa, Anabaena variabilis
QDRYSLR

SEQ ID NO: 33 - protein, 7 aa, Anabaena variabilis
GGNFLGQ

SEQ ID NO: 34 - protein, 7 aa, Anabaena variabilis
EQFNQNI

SEQ ID NO: 35 - protein, 8 aa, Pseudomonas putida
YGINTGFG

SEQ ID NO: 36 - protein, 5 aa, Pseudomonas putida
LVLSH

SEQ ID NO: 37 - protein, 9 aa, Pseudomonas putida
SVGASGDLA

SEQ ID NO: 38 - protein, 7 aa, Pseudomonas putida
QDPYSLR

SEQ ID NO: 39 - protein, 7 aa, Pseudomonas putida
GGNFHAE

SEQ ID NO: 40 - protein, 7 aa, Pseudomonas putida
SANQEDH